(19) United States
(12) Patent Application Publication
(10) Pub. No.: US 2022/0235421 A1
(43) Pub. Date: Jul. 28, 2022

(54) DETECTING AND TREATING CISPLATIN SENSITIVE CANCER

(71) Applicant: The Cleveland Clinic Foundation, Cleveland, OH (US)

(72) Inventors: Jacob Scott, Cleveland, OH (US); Jessica Scarborough, Cleveland, OH (US); Andrew Dhawan, Cleveland, OH (US)

(21) Appl. No.: 17/587,410

(22) Filed: Jan. 28, 2022

Related U.S. Application Data
(60) Provisional application No. 63/274,338, filed on Nov. 1, 2021.
(60) Provisional application No. 63/142,764, filed on Jan. 28, 2021.

(51) Int. Cl. C12Q 1/6886; G01N 33/50; A61K 33/243

(52) U.S. Cl. CPC C12Q 1/6886; A61K 33/243; G01N 33/5011

(57) ABSTRACT

Provided herein are compositions, systems, and methods for prediction of a chemosensitivity to cisplatin or other platinum based drugs for cancer. In certain embodiments, the methods comprise receiving results of, or conducting, an mRNA and/or protein expression level analysis of at least one gene (e.g., 1-13 or 1-19 genes) in a tumor sample from a subject, wherein the gene is expressed at higher levels than a control and is selected from: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750; and treating said subject with Cisplatin or other platinum based cancer drug.

The present application claims priority to U.S. Provisional application Ser. No. 63/142,764 filed Jan. 28, 2021, and 63/274,338 filed Nov. 1, 2021; both of which are herein incorporated by reference in their entireties.

### FIELD OF THE INVENTION

Provided herein are compositions, systems, and methods for prediction of a chemosensitivity to cisplatin or other platinum based chemotherapeutic for treating cancer. In certain embodiments, the methods comprise receiving results of, or conducting, an mRNA and/or protein expression level analysis of at least one gene (e.g., 1-13 or 1-19 genes) in a tumor sample from a subject, wherein the gene is expressed at higher levels than a control (e.g., normal non-cancerous sample) and is selected from: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750; and treating said subject with Cisplatin or other platinum based cancer drug.

### BACKGROUND

Despite rich collections of cancer “-omic” data, precision medicine research has largely focused on producing therapies that target somatic mutations. These therapies have produced some inspiring successes, extending the lives of patients with targetable mutations by months to years. For example, the identification of ALK-mutated tumors has altered the progression non-small cell lung cancer drastically with targeted therapies, such as crizotinib, alectinib, and ceritinib. In its Phase 3 trial, treatment with Crizotinib demonstrated 10.7 month progression-free survival (PFS), while the standard, non-targeted treatment of pemetrexed with a platinum-based agent showed a 7.0 month PFS. However, the reach of genome-driven targeted therapies is narrow and most patients without targetable mutations simply have not seen the benefits of personalized medicine (Ref. 1).

Gene expression signatures are a tool that take advantage of intertumoral heterogeneity without relying on a mutational profile. They can be used to classify, prognosticate, and predict therapeutic response of tumors. A few of these signatures have become invaluable precision medicine tools in the clinic (e.g. OncotypeDxm, Mammaprint) yet a major obstacle in the field is finding gene expression signatures that are robust enough to be predictive in novel datasets. Although there is a great need for distilling complex gene expression data into a clinical tool, most published gene expression signatures perform no better than a null distribution from signatures of the same length, consisting of random genes.

### SUMMARY

Provided herein are compositions, systems, and methods for prediction of a chemosensitivity to cisplatin or other platinum based drug for cancer. In certain embodiments, the methods comprise receiving results of, or conducting, an mRNA and/or protein expression level analysis of at least one gene (e.g., 1-13 or 1-19 genes) in a tumor sample from a subject, wherein the gene is expressed at higher levels than a control and is selected from: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750; and treating said subject with Cisplatin or other platinum based cancer drug.

In some embodiments, provided herein are methods comprising: a) receiving results of (e.g., a lab report over the internet), or conducting, an mRNA or protein expression level analysis of at least one gene from epithelial tumor cells from a subject (e.g., from a tumor, blood sample, cell-line, tissue section, etc.), wherein the at least one gene mRNA and/or protein is expressed at higher levels compared to the at least one gene mRNA and/or protein expression from corresponding non-tumor epithelial cells, wherein the at least one gene is selected from the group consisting of: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750; and b) performing at least one of the following: i) treating the subject with Cisplatin or other platinum based cancer drug, and/or ii) providing a report to the patient or medical personnel treating the patient, indicating the subject is suitable for, or should be, treated with Cisplatin or other platinum based cancer drug.

In certain embodiments, the methods further comprise: receiving results of, or conducting, an mRNA or protein expression level analysis of at least two genes from epithelial tumor cells from a subject, wherein the at least two genes are expressed at higher levels compared the at least two genes from corresponding non-tumor epithelial cells, wherein the at least two genes are selected from the group consisting of: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750. In certain embodiments, the at least two genes is at least three to at least thirteen genes. In further embodiments, the at least two genes is at least thirteen genes that includes all of the following: ADA, NPM3, CSTA, KRT5, KRT14, ATP1B3, USP31, MAP7D3, LRRC8C, C15orf41, LY6K, BNC1, and SLFN11. In additional embodiments, the at least two genes are selected from the group consisting of: NPM3, KRT5, ATP1B3, USP31, LRRC8C, LY6K, and SLFN11. In some embodiments, the at least two genes includes the following 7 genes: NPM3, KRT5, ATP1B3, USP31, LRRC8C, LY6K, and SLFN11. In other embodiments, the at least two genes is at least three to at least nineteen genes. In further embodiments, the at least two genes is at least nineteen genes that includes all of the following: NPM3, KRT5, ATP1B3, USP31, LRRC8C, LY6K, SLFN11, ADAT2, CDIN1, C1QBP, CDC7, CDCA7, FKBP14, MMP10, PSAT1, RIOK1, STOML2, WDR3, and ZNF750.

In some embodiments, the at least one gene comprises: C15orf41, FKBP14, and PSAT1. In other embodiments, the at least the at least one gene comprises: C15orf41, FKBP14, PSAT1, and C1QBP.

In particular embodiments, the subject is a human with cancer. In particular embodiments, the cancer comprises muscle-invasive bladder cancer. In additional embodiments, the method comprises receiving results of conducting an mRNA expression level analysis. In other embodiments, the method comprises conducting an mRNA expression level analysis. In further embodiments, the detecting comprises the use of one or more nucleic acid reagents selected from the group consisting of a nucleic acid primers and nucleic acid probes. In further embodiments, the method comprises conducting protein expression level analysis. In additional embodiments, the detecting comprises the use of one or more antibodies or antigen binding fragments thereof.

In some embodiments, provided herein are kits and systems for detecting altered levels of gene mRNA and/or protein expression in a sample from a subject, comprising: reagents that specifically detect mRNA and/or protein expression from two or more genes selected from the group consisting of: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750. In additional embodiments, the reagents are selected from the group consisting of nucleic acid primers, nucleic acid probes, and antibodies or antigen binding fragments thereof. In further embodiments, the two or more genes comprises: C15orf41, FKBP14, and PSAT1.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A-C shows a schematic representation of Cisplatin Sensitivity Signature derivation. Panel A shows Visual representation of the Genomics of Drug Discovery in Cancer (GDSC) dataset (denoted with the pill icon). These data include 430 epithelial-based cancer cell lines, with drug response to IC50 and gene expression via microarray. Pane B shows a pipeline for extracting connectivity seeds from each fold. First, differential gene expression analysis between the top and bottom 20% of cisplatin responders finds genes with significantly increased expression in a state of cisplatin sensitivity. These differentially expressed genes become “seed genes” in a co-expression network that is used to identify seed genes which are highly co-expressed with each other, denoted as “connectivity genes.” The co-expression network is built using gene expression from clinical samples of epithelial-based tumors in The Cancer Genome Atlas (TCGA). Panel C shows a schematic representation of how the GDSC epithelial-based cancer cell lines from A. are split into 5 folds, where each fold undergoes the pipeline in B. Genes found in at least 3 of the 5 connectivity gene sets are included in the final Cisplatin Sensitivity Signature.

FIG. 2A-D shows Cisplatin Sensitivity Signature attributes. Panel A shows all genes included in the cisplatin sensitivity score along with their abbreviation. Panel B shows an unclustered heatmap showing gene expression of the signature genes in cell lines within the top and bottom quintiles of cisplatin IC50. Color of the heatmap represents the Z-score of gene expression for a given gene (rows) within a given cell line (columns). Cell lines denoted as sensitive (right, yellow bar) tend to display higher expression than cell lines denoted as resistant (left, green bar). Panel C shows distribution of cisplatin IC50 in the 430 epithelial-based GDSC cell lines. Panel D shows distribution of the Cisplatin Sensitivity Signature score in the 430 epithelial-based GDSC cell lines. This score is calculated as a cell line's median expression of the Cisplatin Sensitivity Signature genes listed in A.

FIG. 3 shows cancer subtypes ranked by expression of Cisplatin Sensitivity Signature from Total Cancer Care dataset. A sample's median normalized expression across the 13 cisplatin signature genes is termed the signature score. Violin plots display the distribution of signature scores for each cancer subtype. Within each violin, a boxplot denotes median signature score for each subtype (middle horizontal line) and 25th/75th percentile for signature scores (box edges). The y-axis is labeled by sample TCGA study abbreviations (e.g. LUAD for lung adenocarcinoma). Numbers to the left of each violin plot represent sample size included in each cancer subtype.

FIG. 4 shows correlation between AUC and IC50 drug response metrics for epithelial-based cancer cell lines in the Genomics of Drug Discovery in Cancer (GDSC) dataset.

FIG. 5 shows quality control metrics comparing differential expression results to the final gene signature.

FIG. 6 shows null distribution of Spearman correlation coefficient from regression models built using 1000 random gene signatures. The solid red line represents the median correlation coefficient from all 1000 null models. 95% of the null models' correlation coefficients are between the dashed red lines. The blue line represents the resulting correlation coefficient made by the true signature.

FIG. 7 shows null distribution of AUC values from classification models built using 1000 random gene signatures. The solid red line represents the median AUC value from all 1000 null models. 95% of the null models' AUC values are between the dashed red lines. The blue line represents the resulting AUC from the true signature.

FIG. 8 shows epithelial-based TCGA cancer subtypes ranked by expression of Cisplatin Sensitivity Signature. A sample's median normalized expression across the 13 cisplatin signature genes is termed the signature score. Violin plots display the distribution of signature scores for each cancer subtype. Within each violin, a boxplot denotes median signature score for each subtype (middle horizontal line) and 25th/75th percentile for signature scores (box edges). The y-axis is labeled by sample TCGA study abbreviations (e.g. LUAD for lung adenocarcinoma). Numbers to the left of each violin plot represent sample size included in each cancer subtype.

FIG. 9A-B shows pseudo Kaplan-Meier curves demonstrate differential drug response between cohorts of Cisplatin Sensitivity Signature Expression. Using each cell line's IC50 in place of survival time, these pseudo Kaplan-Meier curves show differences between the top/bottom half and top/bottom quintiles of signature expressing cell lines in A. and B., respectively. In both experiments, the cohort with decreased signature expression tends to have more cell lines with greater IC50 (increased resistance) to cisplatin. A log-rank test is used to statistically compare the “survival curves” of each cohort, with a p-value <0.0001 as denoted in the bottom left of A. and B.

FIG. 10A-C shows Schematic representation of an exemplary CisSig derivation. A. Description of the Genomics of Drug Discovery in Cancer (GDSC) dataset, denoted with the pill icon in future figures. These data include 429 epithelial-based cancer cell lines, with drug response measurements to over 200 drugs and gene expression characterization via microarray. B. Pipeline for extracting connectivity seeds. First, differential gene expression analysis between the top and bottom 20% of cisplatin responders finds genes with significantly increased expression in a state of cisplatin sensitivity. These differentially expressed genes become “seed genes” in a co-expression network built using gene expression from clinical samples of epithelial-based tumors in The Cancer Genome Atlas. Seed genes that are highly co-expressed with each other are denoted as “connectivity genes.” C. Schematic of data partitioning, where GDSC epithelial-based cancer cell lines from A. are split into 5 folds. Each fold undergoes the pipeline in B. Genes found in at least 3 of the 5 connectivity gene sets are included in the final signature, CisSig.

FIG. 11A-D shows Visualization of an exemplary CisSig expression within GDSC Dataset. A. An unclustered heatmap showing gene expression of the CisSig genes (rows) in cell lines (columns) from the top and bottom quintiles of cisplatin IC50. Color of the heatmap represents the Z-score of gene expression, normalized to each gene. Cell lines denoted as sensitive (right, yellow bar) tend to display higher expression of CisSig genes than cell lines denoted as resistant (left, green bar). Z-scores above 2.5 are denoted as 2.5, and Z-scores below −2.5 are denoted as −2.5. B. Violin plots comparing the distribution of CisSig scores between the cell lines in the highest and lowest quintile of cisplatin IC50. C. Comparison of the distribution cisplatin IC50 between cell lines in the highest and lowest quintile of CisSig score. Y-axis represents the proportion of the cohort with a cisplatin IC50 greater than the cisplatin concentration on the X-axis. A log-rank test between the two cohorts demonstrates significantly different drug response between the two cohorts. D. Null distribution of hazard ratio using 1000 random gene signatures with the same length as CisSig and the model described in C. CisSig's performance is compared to the 95% confidence interval of the null distribution, where each signature's performance (CisSig and nulls) is represented by the hazard ratio between two cohorts separated by the signature score.

FIG. 12A-I shows CisSig predicts IC50 using a variety of modeling techniques in the GDSC dataset. A., D., G. Each subplot represents the modeling method for the respective following two plots. For example, A. represents the modeling method used in B. and C. B., E., H. Each subplot represents validation of the best performing cross-validation fold for each respective model. Linear regression models (A. and D.) show a scatter plot comparing predicted vs. actual IC50), while classification models (G.) are represented by a receiver operating characteristic (ROC) curve. C., F., I. Each subplot represents the null distribution of modeling metrics using 1000 random gene signatures with the same length as CisSig for each respective model. CisSig's performance is compared to the 95% confidence interval of the null distribution, where each signature's performance (CisSig and nulls) is represented by the Spearman correlation coefficient or AUC of the best performing fold.

FIG. 13 shows Cancer subtypes with greater CisSig expression tend to have cisplatin included in standard of care guidelines. Cancer subtypes are ranked by median CisSig Score in three data sets, GDSC (left), TCGA (middle), and TCC (right). The ranks of intersecting subtypes between each dataset are compared with a spearman correlation, reported with correlation p and p-value. Violin plots display the distribution of CisSig scores for each cancer subtype. Within each violin, a boxplot denotes median signature score for each subtype (middle horizontal line) and 25th/75th percentile for signature scores (box edges). Numbers to the left of each violin plot represent sample size included in each cancer subtype.

FIG. 14 shows a correlation between AUC and IC50 drug response metrics for epithelial-based cancer cell lines in the Genomics of Drug Discovery in Cancer (GDSC) dataset.

FIG. 15A-B shows CisSig score and cisplatin IC50 are relatively normally distributed in GDSC epithelial cell lines. A. Distribution of CisSig across 429 epithelial-based GDSC cell lines, using a histogram (gray) and kernel density estimation (blue). Median score marked by red vertical line. CisSig score is calculated as a cell line's median normalized expression of CisSig genes listed in A. B. Distribution of cisplatin IC50 across 429 epithelial-based GDSC cell lines, using a histogram (gray) and kernel density estimation (blue). Median IC50 marked by red vertical line.

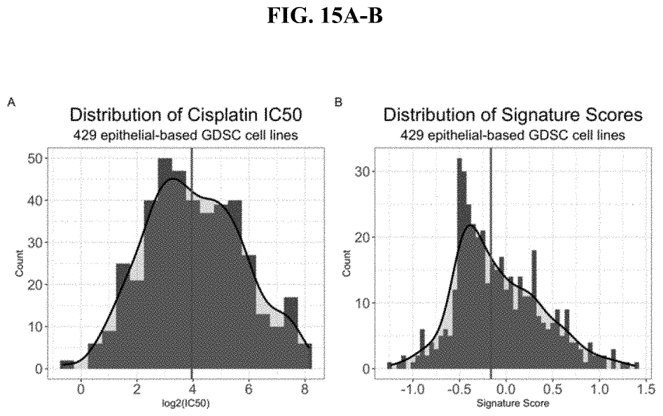

FIG. 16 shows Quality control metrics comparing differential expression results to the final gene signature using sigQC.

FIG. 17A-L: Modeling IC50 response using CisSig Score to predict IC50 in GDSC with simple linear regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 18A-L: Modeling IC50 response using individual CisSig genes to predict IC50 in GDSC with elastic net penalized linear regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 19A-L: Modeling IC50 response using individual CisSig genes to predict IC50 in GDSC with L1 penalized linear regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 20A-L: Modeling IC50 response using individual CisSig genes to predict IC50 in GDSC with L2 penalized linear regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 21A-L: Modeling IC50 response using CisSig score to predict IC50 class in GDSC with simple logistic regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 22A-L. Modeling IC50 response using individual CisSig genes to predict IC50 class in GDSC with elastic net penalized logistic regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 23A-L: Modeling IC50 response using individual CisSig genes to predict IC50 class in GDSC with L1 penalized logistic regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 24A-L: Modeling IC50 response using individual CisSig genes to predict IC50 class in GDSC with L2 penalized logistic regression. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 25A-L: Modeling IC50 response using individual CisSig genes to predict IC50 class in GDSC with support vector machine modeling (linear kernel). A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 26A-L: Modeling IC50 response using individual CisSig genes to predict IC50 class in GDSC with support vector machine modeling (polynomial kernel). A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 27A-L: Modeling IC50 response using individual CisSig genes to predict IC50 class in GDSC with random forest modeling. A-E. Predicted vs. Actual IC50 for validation sets of folds 1-5 for models built with all 429 cell lines. F. Null distribution of modeling metrics using 1000 random gene signatures with the same length as CisSig and the model described in A-E. CisSig's performance (blue line) is compared to the 95% confidence interval (red dashed lines) of the null distribution. Red line represents median of null distribution. G-K. Predicted vs. Actual IC50 for validation sets of folds 1-5 for models built using cell lines in the top and bottom 20% of cisplatin IC50. F. Null distribution of modeling metrics using 1000 random gene signatures with the same length as CisSig and the model described in G-K. CisSig's performance (blue line) is compared to the 95% confidence interval (red dashed lines) of the null distribution. Red line represents median of null distribution.

FIG. 28A-G: CisSig-trained model is predictive in patients who have received cisplatin, but lacks signal in patients who have not received cisplatin. A. Schematic description of model training and testing, where model is trained using patients who did receive cisplatin-containing treatment from Dataset A dataset. Testing of the trained model is done using patients from the Dataset A who did not receive cisplatin-containing treatment and patients from the Dataset B dataset who did receive cisplatin-containing treatment. B. Test samples that did receive cisplatin-containing treatment are separated into groups of “high” and “low risk” based on the model's predictions using a median cutoff. Kaplan-meier curves show a significant separation between the two groups. C. The same analysis shown in B, using an optimal cutpoint instead of median to separate the cohorts. D-E. The same analyses shown in B-C, separating the groups into “high”, “middle”, and “low risk” groups using tertiles and an optimal two cutpoints, respectively. F-G. The same analyses shown in B and D, using samples from Dataset A that did not receive cisplatin-containing treatment, demonstrating no significant separation between the two groups.

FIG. 29A-G: CisSig-trained model is predictive in patients who have received cisplatin, but lacks signal in patients who have not received cisplatin. A. Schematic description of model training and testing, where model is trained using patients who did receive cisplatin-containing treatment from Dataset B dataset. Testing of the trained model is done using patients from the Dataset A who did not receive cisplatin-containing treatment and patients from the Dataset A dataset who did receive cisplatin-containing treatment. B. Test samples that did receive cisplatin-containing treatment are separated into groups of “high” and “low risk” based on the model's predictions using a median cutoff. Kaplan-meier curves show a significant separation between the two groups. C. The same analysis shown in B, using an optimal cutpoint instead of median to separate the cohorts. D-E. The same analyses shown in B-C, separating the groups into “high”, “middle”, and “low risk” groups using tertiles and an optimal two cutpoints, respectively. F-G. The same analyses shown in B and D, using samples from Dataset A that did not receive cisplatin-containing treatment, demonstrating no significant separation between the two groups.

### DETAILED DESCRIPTION

Provided herein are compositions, systems, and methods for prediction of a chemosensitivity to cisplatin. In certain embodiments, the methods comprise receiving results of, or conducting, an mRNA and/or protein expression level analysis of at least one gene (e.g., 1-13 genes) in a tumor sample from a subject, wherein the gene is expressed at higher levels than a control and is selected from: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750; and treating said subject with Cisplatin or other platinum based cancer drug.

The present invention is not limited to particular methods of detecting the level of the recited gene markers (e.g., in Table 10 and throughout). Markers may be detected as DNA (e.g., cDNA), RNA (e.g., mRNA), or protein.

In some embodiments, nucleic acid sequencing methods are utilized for detection. In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to cDNA before sequencing.

A number of DNA sequencing techniques are suitable, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In some embodiments, hybridization methods are utilized. Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with radio-, fluorescent- or antigen-labeled bases is localized and quantitated in the tissue using autoradiography, fluorescence microscopy or immunohistochemistry. ISH can also use two or more probes, labeled with radioactivity or the other non-radioactive labels, to simultaneously detect two or more transcripts.

In some embodiments, markers are detected using fluorescence in situ hybridization (FISH). The preferred FISH assays for methods of embodiments of the present disclosure utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

Different kinds of biological assays are called microarrays including, but not limited to: microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Southern and Northern blotting may be used to detect specific DNA or RNA sequences, respectively. In these techniques DNA or RNA is extracted from a sample, fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

In some embodiments, marker sequences are amplified (e.g., after conversion to DNA) prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, quantitative evaluation of the amplification process in real-time is performed. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In certain embodiments, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782, each of which is herein incorporated by reference in its entirety.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

The marker genes described herein may be detected as proteins using a variety of protein techniques known to those of ordinary skill in the art, including but not limited to: protein sequencing; and, immunoassays. Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

Embodiments of the present invention further provide kits and systems comprising reagents for detection of the recited markers (e.g., primer, probes, etc.). In some embodiments, kits and systems comprise computer systems for analyzing marker levels and providing diagnoses, prognoses, or determining treatment courses of action (e.g., indicating if the subject should be treated with Cisplatin or not, or with some other platinum based drug or not).

In some embodiments, a computer-based analysis program is used to translate the raw data generated by the detection assay (e.g., mRNA or protein levels of the recited markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject (e.g., if the subject should be treated with Cisplatin or not, or with some other platinum based drug or not).

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced (i.e., marker levels) specific for the diagnostic or prognostic information desired for the subject (e.g., producing a report that indicates if the subject should be treated with Cisplatin or not, or with some other platinum based drug or not).

The profile data is then prepared in a format (e.g., electronic or printed report) suitable for interpretation by a treating clinician. For example, rather than providing raw data, the prepared format may represent a diagnosis or risk assessment (e.g., level of markers) for the subject, along with recommendations for particular treatment options (e.g., indicating if the subject should be treated with Cisplatin or not, or with some other platinum based drug or not) The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject or medical care provider is able to directly access the data using the electronic communication system. The subject may chose further intervention or counseling based on the results.

### EXAMPLES

### Example 1

### Cisplatin Gene Signature

### Genomics of Drug Sensitivity in Cancer (GDSC) Dataset Description

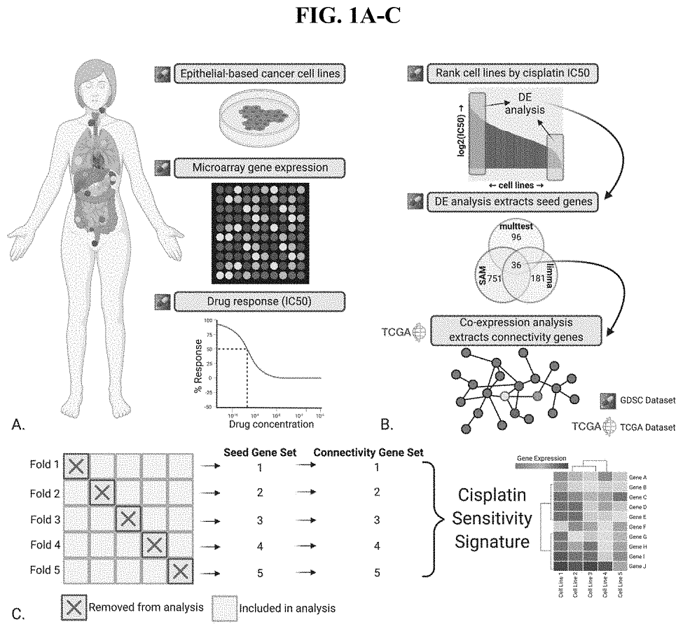

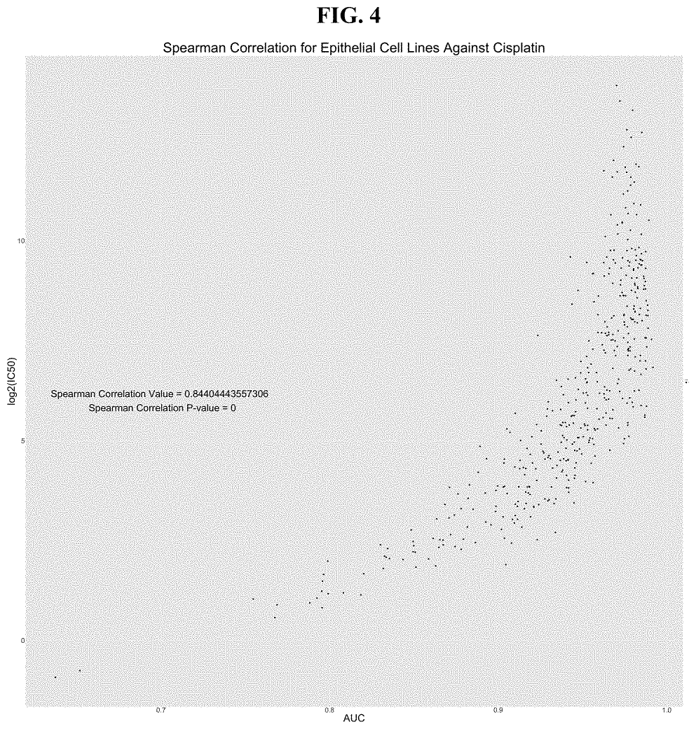

RMA normalized microarray mRNA expression, drug response, and meta-data for 983 cell lines and 251 drugs was downloaded from the Genomics in Drug Sensitivity Database (GDSC) (Yang. 2013). The GDSC dataset contains 430 epithelial-based cancer cell lines which have been tested against cisplatin, visually represented in FIG. 1, panel A. This dataset contains both AUC and IC50 metrics to summarize the drug response of each cell line. Although IC50 is traditionally used for reporting sensitivity to a drug, we recognize that this measure is not always representative of true drug response. Before moving forward with IC50 as our chosen drug response metric, we compared IC50 and AUC values of the 430 cell lies of interest to ensure moderate agreement between the two variables shown in FIG. 4. This analysis produced a Spearman correlation coefficient of 0.84 and a p-value <<0.001, which demonstrates that using the IC50 does not differ significantly from AUC when ranking cell lines by their response to cisplatin.

The GDSC epithelial cell lines were split into five folds (containing 344 cell lines), each with a different 20% of the cell lines removed, illustrated in FIG. 1, panel C. Each of these folds were analyzed with a pipeline of differential gene expression and co-expression analysis, visually depicted in FIG. 1, panel B. This step was performed over folds of the data in order to find genes that are consistently important between folds, reducing the chance for outlier cell lines to influence the results.

Seed genes are extracted using DE analysis to compare cisplatin-sensitive and -resistant cell lines. Cell lines with the highest and lowest 5% of IC50 values in each fold were removed in order to decrease the incidence of poorly modeled extreme drug responses from our analysis. Then, differential gene expression (DE) analysis using limma, SAM, and methods was performed between the top and bottom 20% of responders (i.e. cell lines with the highest and lowest 20% of IC50 values). Each comparison group contained 62 cell lines. For each fold, the genes over-expressed in a cisplatin-sensitive state by all three DE methods were termed the “seed genes,” resulting in 5 sets of seed genes, as depicted in FIG. 1, panel C. Tables containing the number of significantly differentially expressed genes found from each fold's DE analysis may be found in Tables 2-6.

TABLE 2
Number of differentially expressed genes found in fold 1.

| Method | Num. Up-regulated Genes | Num. Down-regulated Genes |
|---|---|---|
| SAM | 751 | 549 |
| limma | 181 | 244 |
| multtest | 96 | 89 |
| intersection | 36 | 28 |

TABLE 3
Number of differentially expressed genes found in fold 2.

| Method | Num. Up-regulated Genes | Num. Down-regulated Genes |
|---|---|---|
| SAM | 744 | 568 |
| limma | 191 | 245 |
| multtest | 1832 | 1850 |
| intersection | 135 | 151 |

TABLE 4
Number of differentially expressed genes found in fold 3.

| Method | Num. Up-regulated Genes | Num. Down-regulated Genes |
|---|---|---|
| SAM | 863 | 624 |
| limma | 197 | 244 |
| multtest | 117 | 92 |
| intersection | 37 | 25 |

TABLE 5
Number of differentially expressed genes found in fold 4.

| Method | Num. Up-regulated Genes | Num. Down-regulated Genes |
|---|---|---|
| SAM | 1033 | 707 |
| limma | 209 | 259 |
| multtest | 1987 | 1631 |
| intersection | 171 | 179 |

TABLE 6
Number of differentially expressed genes found in fold 5.

| Method | Num. Up-regulated Genes | Num. Down-regulated Genes |
|---|---|---|
| SAM | 290 | 344 |
| limma | 161 | 229 |
| multtest | 55 | 51 |
| intersection | 15 | 18 |

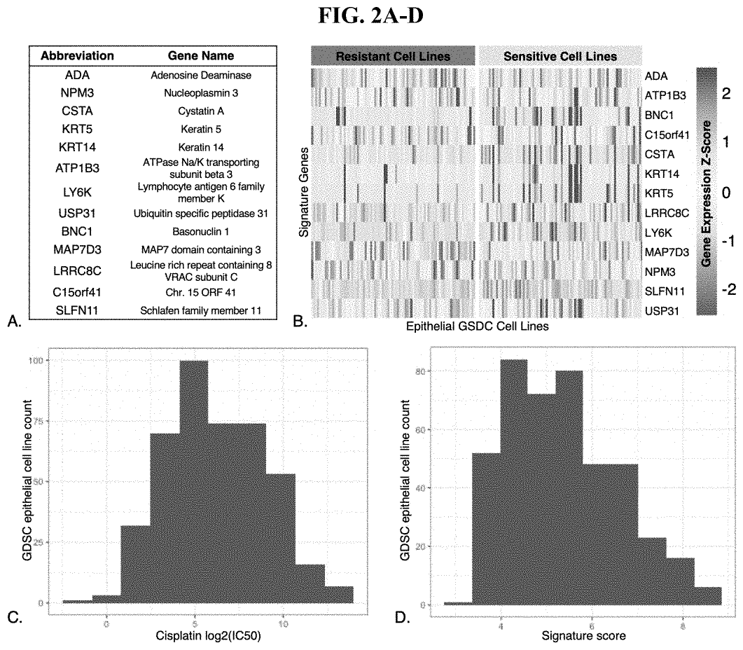

Seed genes are used to build co-expression networks, which inform the final signature. A co-expression network was built for each set of seed genes, as described in Methods, and visually represented in the bottom panel of FIG. 1, panel B. These networks were built using TCGA RNA-Seq expression data and compared the expression of seed genes and all other genes in the dataset. Using this network analysis, seed genes that were highly co-expressed with each other are extracted from each fold, termed “connectivity seeds.” As shown in FIG. 1, panel C, this is performed for each set of seed genes and produces 5 sets of connectivity seeds. The final gene signature contains any gene found in at least 3 of the 5 sets of connectivity seeds, and the genes included in the final signature are listed in FIG. 2, panel A.

TABLE 7
Genes included in cisplatin sensitivity gene expression signature.

| Entrez ID | Gene Symbol | Gene Name. |
|---|---|---|
| 100 | ADA | Adenosine Deaminase |
| 10360 | NPM3 | Nucleoplasmimn 3 |
| 1475 | CSTA | Cystatin A |
| 3852 | KRT5 | Keratin 5 |
| 3861 | KRT14 | Keratin 14 |
| 483 | ATP1B3 | ATPase Na+/K+ transporting subunit beta 3 |
| 54742 | LY6K | Lymphocyte antigen 6 family member K |
| 57478 | USP31 | Ubiquitin specific peptidase 31 |
| 646 | BNC1 | Basonuclin 1 |
| 79649 | MAP7D3 | MAP7 domain containin 3 |
| 84230 | LRRC8C | Leucine rich repeat containing 8 VRAC |
| | | subunit C |
| 84529 | C15orf41 | Chr. 15 ORF 41 |
| 91607 | SLFN11 | Schlafen family member 11 |

A co-expression network was built for each fold's DE genes, using TCGA RNA-seq expression data from 7432 epithelial-based cancer samples. Each co-expression network was built from a pairwise comparison between expression of a fold's “seed genes” and all genes in the dataset. The value of this pairwise comparison was termed the “affinity score” between the two genes. The affinity scores were ranked and underwent a binary transformation, where the scores in the bottom 95th percentile were converted to 0 and those in the top 5th percentile were converted to 1. Then, the average affinity of each gene to all of the seed genes is found, termed the “connectivity score.” All genes were ranked by their connectivity score. The intersection of the differentially expressed seed genes and the top 15% of the genes ranked by connectivity score is extracted. For each fold, these overlapping genes were termed the “connectivity seeds.” The final gene signature contains any gene found in at least 3 of the 5 sets of connectivity seeds.

FIG. 2, panel B demonstrates the expression of the Cisplatin Sensitivity Signature genes in cisplatin-sensitive and -resistant GDSC cell lines. From this, we see that signature expression tends to be higher in sensitive, rather than resistant, cell lines. This is expected, because the seed genes were initially extracted as genes with increased expression in a cisplatin-sensitive state in the GDSC dataset. FIG. 2, panel C shows the distribution of cisplatin IC50 values in all GDSC cell lines included in the analysis. Finally, a cell line or sample's median expression value for the 13 signature genes is termed its “signature score.” The distribution of these signature scores across the same GDSC cell lines is displayed in FIG. 2, panel D. Comparing FIG. 2, panel C and panel D, we can observe that the distribution of signature score and drug response are relatively similar.

The Cisplatin Sensitivity Signature outperforms the null distributions of drug response prediction models. To explore the predictive abilities of the Cisplatin Sensitivity Signature, various models were built to predict cisplatin response of the GDSC epithelial-based cell lines using five-fold cross validation. For each fold, each model was built twice, once with all GDSC data and again using only cell lines found in the top and bottom quintiles of signature scores. Using both dataset versions allowed us to interrogate whether more extreme signature scores tend to predict cisplatin response with improved accuracy.

Simple linear regression and L2-penalized linear regression models used signature score and expression of all signature genes, respectively, to predict a cell line's IC50 as a continuous variable. Each model's performance is compared using the Spearman correlation coefficient between the predicted and actual IC50 value for the cell lines withheld from a given fold's training dataset. The best correlation coefficient between the five folds is chosen to represent each model, shown in Table 8.

TABLE 8
Validating models built for cisplatin response prediction using Cisplatin Sensitivity
Score in GDSC dataset.

| Input | Output | Method | Included Data | Metric | Value |
|---|---|---|---|---|---|
| Signature Score | IC50 (continuous) | Simple linear Regression | All | Corr. Coef. | 0.41 |
| Signature Score | IC50 (continuous) | Simple linear Regression | Quintiles | Corr. Coef. | 0.68 |
| All gene expression | IC50 (continuous) | L2-Penalized Linear Regression | All | Corr. Coef. | 0.57 |
| All gene expression | IC50 (continuous) | L2-Penalized Linear Regression | Quintiles | Corr. Coef. | 0.62 |
| All gene expression | Binary | L2-Penalized Logistic Regression | All | AUC | 0.79 |
| All gene expression | Binary | L2-Penalized Logistic Regression | Quintiles | AUC | 0.80 |
| All gene expression | Binary | Support Vector Machine | All | AUC | 0.74 |
| All gene expression | Binary | Support Vector Machine | Quintiles | AUC | 0.85 |
| All gene expression | Binary | Random Forest | All | AUC | 0.77 |
| All gene expression | Binary | Random Forest | Quintiles | AUC | 0.83 |

There, we see that both models demonstrate improved performance when trained and tested on cell lines with the highest and lowest signature scores (quintiles). Additionally, when trained with all cell lines, the L2-penalized linear regression model greatly outperforms the simple linear regression model. When trained with just the cell lines in the top and bottom quintiles, their performance is comparable, with simple linear regression slightly outperforming the L2-penalized linear regression.

L2-penalized logistic regression, support vector machine, and random forest models use the expression of each signature gene to predict a cell line's IC50 as a binary outcome (IC50 above or below the median). Additional details regarding the implementation of these models can be found in the Methods Section. We use area under the ROC curve (AUC) to represent each of the classification model's performance.

Again, the best AUC value between the five folds is chosen to represent the model, listed in Table 1. Here, we see that the three classification models had comparable performances, measured by AUC. And similar to the linear regression models, models trained and tested on cell lines with the highest and lowest signature scores have improved performance.

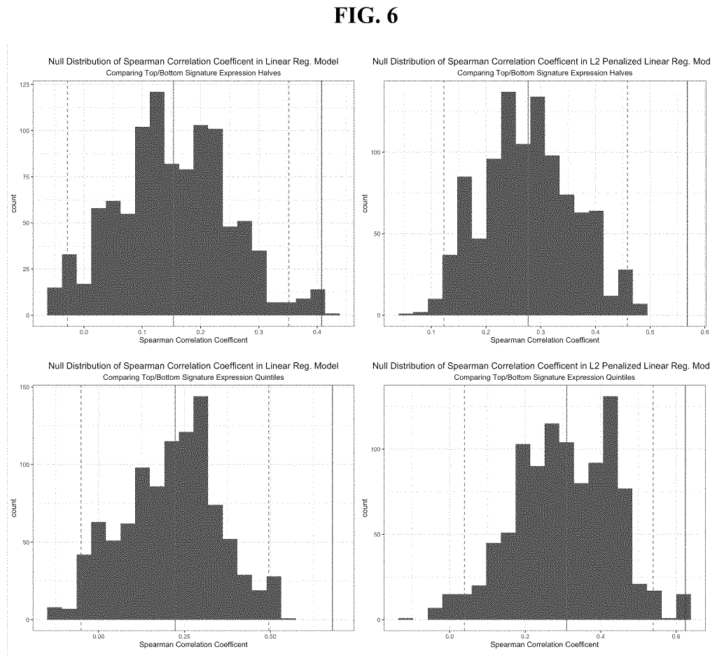

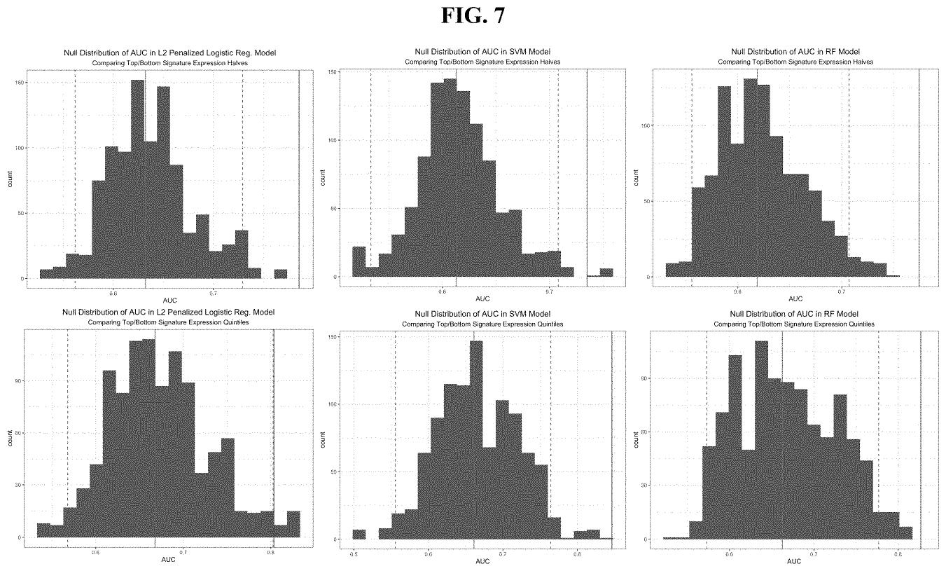

For each model built using the Cisplatin Sensitivity Signature, a null distribution was also produced. This was done using 1000 random gene signatures each with 13 genes, the same number of genes included in the signature in question. FIGS. 6 and 7 show that the Cisplatin Sensitivity Signature performs in the top 2.5th percentile or higher of the null distributions for each model's summary statistic. Overall, we see that our gene expression signature has significant predictive capabilities in the setting of many different models.

Differential distribution of IC50 is visualized by signature expression cohort. In order to visualize the differential distribution of IC50 values by signature expression, we compared the fraction of cell lines with greater than a certain IC50 for cell lines with high and low signature expression. These curves resemble Kaplan-Meier survival curves, but use IC50 in place of survival time.

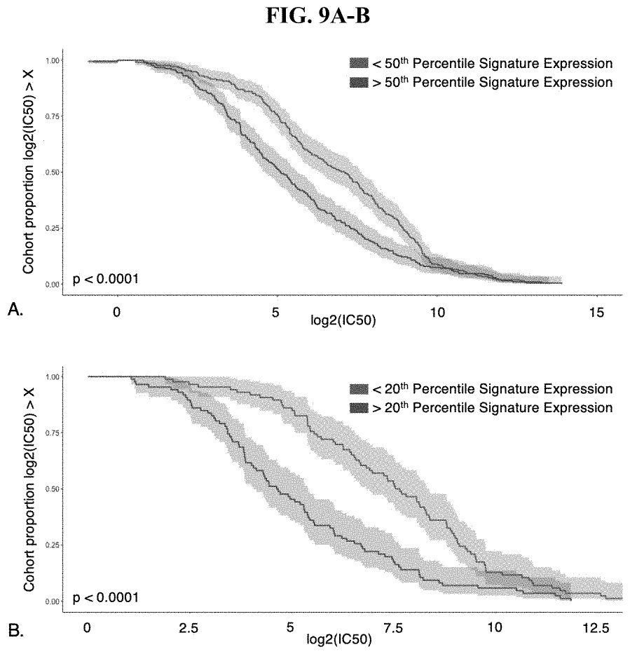

This analysis is performed twice; once, comparing cell lines with the top and bottom halves of signature expressors and, again, with cell lines in the top and bottom quintiles of signature expressors. FIG. 9 demonstrates that the two cohorts of expressors contain significantly different IC50 values, each with a p<0.0001 when compared with a log-rank test. The low signature expression cohort contains more cell lines with greater IC50 values than the cohort of high signature expression. This is consistent with the hypothesis that high expression of the Cisplatin Sensitivity Signature tends to correlate with a lower IC50 (greater sensitivity) in a given cell line or sample.

Cancer Subtypes from Independent Clinical Data are Ranked by Signature Expression.

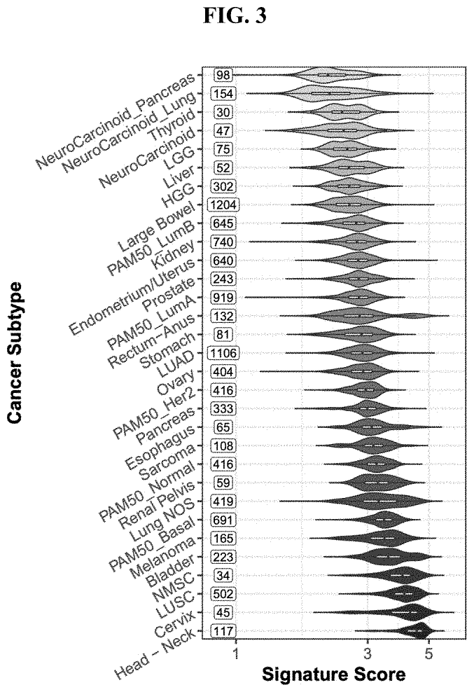

In order to examine expression of the Cisplatin Sensitivity Signature in independent clinical samples, a signature score was calculated for all Total Cancer Care clinical samples of epithelial origin. First, gene expression for all genes underwent z-score normalization, then each sample's median expression of the 13 signature genes was extracted. In order to visualize these scores on a log-transformed axis, signature score was linearly scaled, making the lowest score exactly 1. Cancer subtypes were ranked by the median signature score for the samples in each group, seen in FIG. 3.

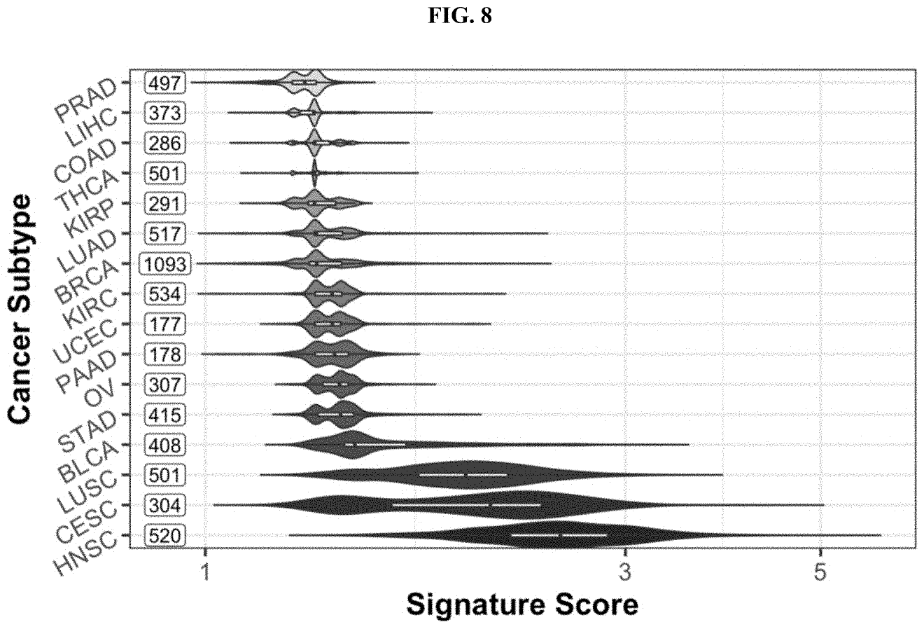

The same analysis was performed in the TCGA dataset, with ranked cancer subtypes shown in FIG. 8. In both datasets, we see that median signature scores were highest for head-neck squamous cell carcinoma and cervical cancer. Cancers that are less frequently treated with cisplatin (e.g. thyroid, colon cancer) tend to have lower expression of the cisplatin sensitivity signature. These findings demonstrate that the Cisplatin Sensitivity Signature tends to correlate with current clinical practices.

sigQC is Used to Analyze the Quality of the Cisplatin Sensitivity Signature in a Clinical Dataset.

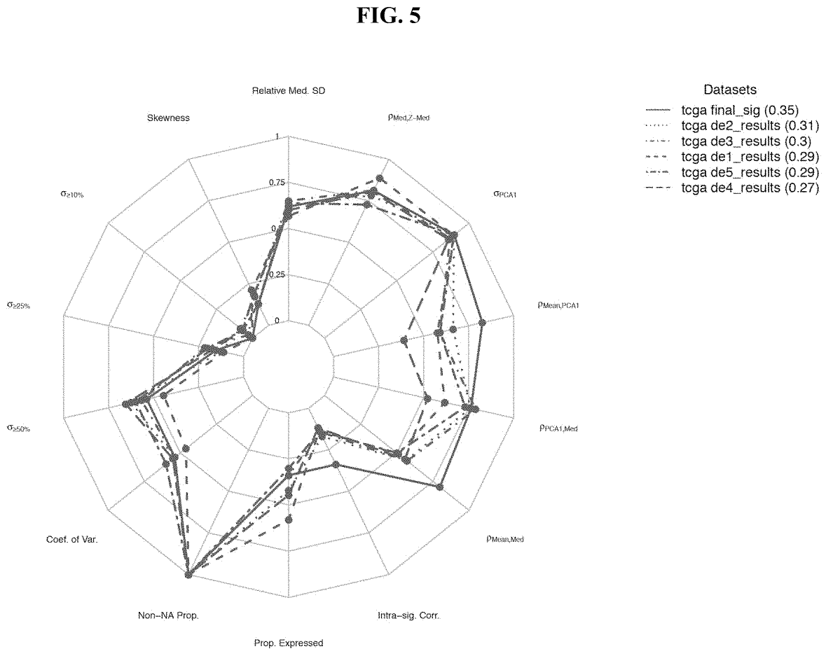

Using the sigQC package in R, we analyzed a suite of quality control metrics to assess the robustness of the Cisplatin Sensitivity Signature in a clinical sample (TCGA) dataset. The signature is compared to the 5 sets of seed genes originally extracted from GDSC, prior to being trimmed with co-expression analysis. These results are visualized in a radar plot in FIG. 5. The Cisplatin Sensitivity Signature demonstrates greater intra-signature correlation, increased correlation between mean and median, and decreased skewness within RNA-expression from TCGA samples of epithelial origin. Other metrics of interest include the coefficient of variance and the proportion (sigma) of signature genes found in the top 10%, 25% or 50% of variable genes. These metrics can be used to assess the variability of signature genes within a dataset. It is ideal to have signature genes that vary more than the background noise of the dataset. Here, the Cisplatin Sensitivity Signature performs similarly to the unfiltered differential gene expression results. Finally, these metrics are summarized into a score, also displayed in FIG. 5, where the Cisplatin Sensitivity Signature outperformed all of the differential expression results alone.

In this work, we demonstrate a novel method for empirically deriving gene expression signatures, producing a Cisplatin Sensitivity Signature. Despite being used for drug response prediction, this method can be generalized to create signatures that predict any quantitative or binary phenotypic outcome. Here, epithelial-based GDSC tumor cell lines were ranked by their response to a cisplatin and the best and worst responders were extracted for differential gene expression analysis with GDSC expression data. Genes with increased expression in the treatment sensitive state were used as seeds in a co-expression network built from expression data from a disparate clinical dataset, TCGA).

The final gene signature was formed by extracting seed genes that were also highly co-expressed within the TCGA dataset. By ensuring that signature genes associated with cisplatin sensitivity in the GDSC dataset (seed genes) and co-expressed in TCGA expression data, we expect that our gene signature will have improved performance in novel datasets. This is especially important, because the GDSC dataset provides drug response and expression data of cell lines, which have been notoriously difficult to translate to clinical (Refs. 12 and 13).

As demonstrated by many predictive models that were built and validated, our gene signature has significant predictive capabilities in the GDSC dataset, from which it was originally derived. The Cisplatin Sensitivity Signature demonstrated significant predictive capabilities in the GDSC dataset, from which it was originally derived. Table 1 shows that a variety of predictive models can successfully utilize the Cisplatin Sensitivity Signature to predict drug response. Regression models have correlation values between [0.41-0.68] between the testing dataset's predicted and actual values, while classifiers demonstrate an AUC range of [0.74-0.85] in the testing dataset. Most importantly, a null distribution for each model's performance was built using 1000 random gene expression signatures comprised of the same number of genes as found in the Cisplatin Sensitivity Signature. The Cisplatin Sensitivity Signature outperforms the 95% confidence interval for each model's null distribution.

Validation with an independent dataset is crucial for assessing the translational value of the Cisplatin Sensitivity Signature. Examining how the signature performs within an independent clinical dataset, TCC, provides an independent metric of validation. However, no large clinical sample database contains drug response data for the tumor samples, which means modeling which uses expression of the signature genes to predict treatment response cannot be validated in these datasets because the “true” drug response values for each sample are not known. FIG. 4 demonstrates cancer subtypes (from Moffit's TCC samples) ranked by median signature score of each group. There, we see that cancer subtypes frequently treated with cisplatin (e.g. head and neck, cervical) tend to have greater Cisplatin Sensitivity Signature scores. Even without performing predictive modeling with this independent dataset, we can see that this gene expression signature tends to be congruent with current clinical practices.

This Cisplatin Sensitivity Signature can be useful in many clinical and research circumstances. There are various cancer diagnoses with multiple “gold-standard” therapies. In these scenarios, physician or institutional preference may be the only deciding factor in which treatment option a patient should receive. If some (but not all) of the options contained cisplatin, this signature could help inform the decision regarding which option is best, bringing personalized medicine to the many cancer patients who do not have targetable mutations. Finally, clinical trials may use these types of signatures to stratify patients by predicted response to cisplatin, helping account for heterogeneity in clinical outcomes.

### Data Collection and Pre-Processing

GDSC Data

Microarray mRNA expression, drug response, and meta-data for 983 cell lines and 251 drugs was downloaded from the Genomics in Drug Sensitivity Database (GDSC) (Ref. 6). The expression and meta-data was last updated 4 Jul. 2016. The drug-response data was last updated 27 Mar. 2018; this version of the drug response data is referred to as “GDSC2.” The GDSC database can be accessed at https://www.cancerrxgene.org/.

Documentation for the GDSC database states that the RMA normalized (Ref. 14) expression data for all cell lines were collected via Affymetrix Human Genome U219 Array. The raw data and probe ID mappings were deposited in ArrayExpress (accession number: E-MTAB-3610). The RMA processed dataset is available at www.followed by cancerrxgene.org/gdsc1000/. Raw viability data were processed using the R package, gdscIC50, where they were normalized with negative controls (media alone) and positive controls (media only wells with no cells). Additionally dose-response curves were fit using a multi-level fixed effect model with a classic sigmoidal curve shape assumed. This model was fitted using all cell line/drug combinations that were screened instead of fitting separate models to individual drug-response series. In this approach, the shape parameter only changes between cell lines, but the position parameter is adjusted between cell lines and compounds. Additional information regarding dose-response curve fitting may be found at Vis et al. (Ref. 15). Fitting models to all dose-response series leads to improved robustness for more accurate IC50 and AUC estimates.

Genes from the GDSC database are labeled with Ensembl gene identifiers, while the TCGA database utilizes Entrez gene identifiers. In order to cohesively work between the two datasets, the Ensembl gene identifiers are converted to Entrez gene identifiers using the biomaRt R package (Refs. 16 and 17) on 16 Sep. 2019.

Epithelial-based cell lines are extracted based on the following GDSC tissue descriptors: “head and neck”, “oesophagus”, “breast”, “biliary_tract”, “digestive_system_other”, “large_intestine”, “stomach”, “lung_NSCLC_adenocarcinoma”, “lung_NSCLC_carcinoid”, “lung_NSCLC_large cell”, “lung_NSCLC_not specified”, “lung_NSCLC_squamous_cell_carcinoma”, “Lung_other, pancreas”, “skin_other”, “thyroid”, “Bladder”, “cervix”, “urogenical_system_other”, and “uterus”.

TCGA Data

RSEM normalized gene expression for epithelial-based cancers was downloaded from The Cancer Genome Atlas (TCGA) database, which was accessed through the Firebrowse database at http://www.firebrowse.org. The following TCGA Study Abbreviations were downloaded: “BLCA”, “BRCA”, “CESC”, “COAD”, “HNSC”, “KIRP”, “LGG”, “KIRC”, “LIHC”, “LUAD”, “LUSC”, “OV”, “PAAD”, “PRAD”, “STAD”, “THCA”, and “UCEC.” These values were taken through the Illumina HiSeq RNAseq V2 platform and were log 2 transformed. The expression of all 17 types of epithelial-based cancers is combined, resulting in a dataset that contains genes sequenced in each TCGA set.

Total Cancer Care (TCC) Data

The Total Cancer Care Dataset is collected by the H. Lee Moffitt Cancer Center & Research Institute using protocols described in Fenstermacher et al (Ref. 18).

Drug Response Quality Control

IC50 is an imperfect measure of drug response, yet it is widely used throughout the literature. To increase our confidence that IC50 is an acceptable representation of differences in drug response, the IC50 and AUC values for all epithelial cell lines are compared using a Spearman correlation test. A significant correlation score would confer some credence to the use of IC50 in characterizing the set of cell lines included in the experiment.

Differential Gene Expression Analysis

As seen in FIG. 1 the GDSC dataset is split into 5-folds, where 1 different fold is removed from further analysis for each of the 5 runs. For each run, cell lines are ranked based on their IC50 and the top or bottom 5% are removed from the dataset as quality control, because extreme IC50 estimates may indicate a poorly fit drug-response model. After removing the cell lines with IC50 extremes, the top 20% and bottom 20% are extracted for comparison using differential expression analysis, FIG. 1. Differential expression analysis is performed using three algorithms, significance analysis of microarrays (SAM), resampling-based multiple hypothesis testing, and linear models for microarrays (limma), which are implemented using R packages samr (Ref 8), multtest (Ref 9) and limma (Ref. 7), respectively. This analysis has 62 samples per group, which is appropriate given the demonstration by Baccarella et al. showing that differential expression results can vary widely when there are as few as 8 samples per group (Ref. 19). A false discovery rate of 0.20 was chosen for each method. The intersection of the genes found to have significantly increased expression in sensitive cell lines by the three algorithms is termed “seed genes” for use in future co-expression analysis.

Co-Expression Network Analysis and Final Signature Derivation

The co-expression network, represented in the pipeline of FIG. 1, panel B, is made by performing a pairwise Spearman correlation between the expression of each seed gene and every other gene (including other seed genes). The correlation coefficient for each pairwise comparison is termed the “affinity score.”

### Signature Validation

Modeling for Prediction

A cell line or sample's median expression value of the signature genes is termed the signature score. Cell lines were again organized into five folds (independent from the fold for DE analysis), where each fold has 20% of the cell lines separated as testing data. Predictive models were trained from each of the five folds and testing using each fold's respective testing dataset. All models were built with two datasets—one using all of the epithelial-based cell lines and the other using only the cell lines in the top and bottom quintiles of for signature score. When using all the epithelial-based cell lines, training sets consist of 344 cell lines, while testing sets consist of 86 cell lines. When using only the cell lines in the top and bottom quintiles for signature expression, training sets consist of 137 or 138 cell lines and testing sets consist of 34 or 35 cell lines.

Linear regression was used to predict IC50 given a cell line's signature score. L2-penalized linear regression utilized the expression of each of the 13 signature genes to predict IC50. Both linear regression models were evaluated using the Spearman correlation coefficient between true and predicted IC50 values. L2-penalized logistic regression, support vector machine, and random forest models were fit to predict whether a cell line would be in the top half or bottom half of IC50. Support vector machines had a polynomial kernel, and each model was tuned to choose the best options between degree (3, 4, 5), gamma (10⁻⁵, 10⁻⁴, 10⁻³, 10⁻², 10⁻¹), and cost (−3, −2, −1, 0, 1, 2, 3). The random forest model grew 500 trees. All other parameters in training the prediction models were default. The code for building all of these models can be viewed in the previously described GitHub repository. Each of these classification algorithms was evaluated using the area under the curve (AUC) value for the receiver operating characteristics (ROC) curve for each model.

For each model, 1000 random gene signatures of the same length are tested to produce a null distribution of these summary statistics. The null models are built using random gene signatures of the same length as the Cisplatin Sensitivity Signature (13 genes). Just as seen in the true gene signature, each null gene signature is tested using five-fold cross validation for each model of interest and the best summary statistic of the five-folds is chosen to represent the signature's performance with a given model. All code for building the testing and null models may be found in the previously described GitHub repository.

Pseudo-Kaplan-Meier Statistics

Cell lines with high signature scores (predicting the more sensitive cell lines) and low signatures scores (predicting the more resistant cell lines) are separated. A Kaplan-Meier survival model is built using IC50 scores for epithelial-based cell lines in lieu of survival time. Again, two of these models were built, once using cell lines in the top and bottom half of signature scores and again using cell lines in the top and bottom quintiles of signature scores. A log-rank test is performed to analyze if the two cohorts of signature expression are related to different “survival” of higher IC50s in each group.

Signature Quality Control in TCGA

In order to examine how the gene signature compares to the original differential gene expression results, we perform a quality control analysis within the TCGA dataset using the sigQC R package (Refs. 10 and 11). Here, various metrics are calculated using the expression of the genes found in the gene expression signature and the 5 sets of differential expression analysis results. These metrics include intra-signature correlation, correlation between the mean expression and first principal component, and skewness of the signature expression. The final results of all the metrics calculated for each signature are displayed in a radar plot, with a summary score of each set of genes (signature) tested.

### Example 2

### Additional Cisplatin Gene Signature

The approach in this Example empirically derives seed genes using differential gene expression analysis, comparing cisplatin-sensitive and resistant cell lines from the Genomics of Drug Sensitivity in Cancer (GDSC) database. This evolutionary-inspired approach exploits the principles of convergent evolution, where genomically disparate organisms (a variety of cancer subtypes) evolve similar phenotypes (cisplatin response) independently. With cisplatin acting as a selecting agent, natural selection acts on the phenotype of cisplatin response. Our method aims to find predictable patterns of gene expression to relate to this crucial tumor characteristic.

These differentially expressed seed genes are trimmed based on co-expression in epithelial-based tumor samples from The Cancer Genome Atlas. With this final signature, we demonstrate that Cisplatin Sensitivity Signature (CisSig) is highly predictive of cisplatin response within the original cell line dataset (GDSC). And finally, we establish that CisSig expression in independent datasets of clinical tumor samples is congruent with use of cisplatin in standard of care guidelines between disease sites.

Results

CisSig is Derived from the Genomics of Drug Sensitivity in Cancer (GDSC) Database

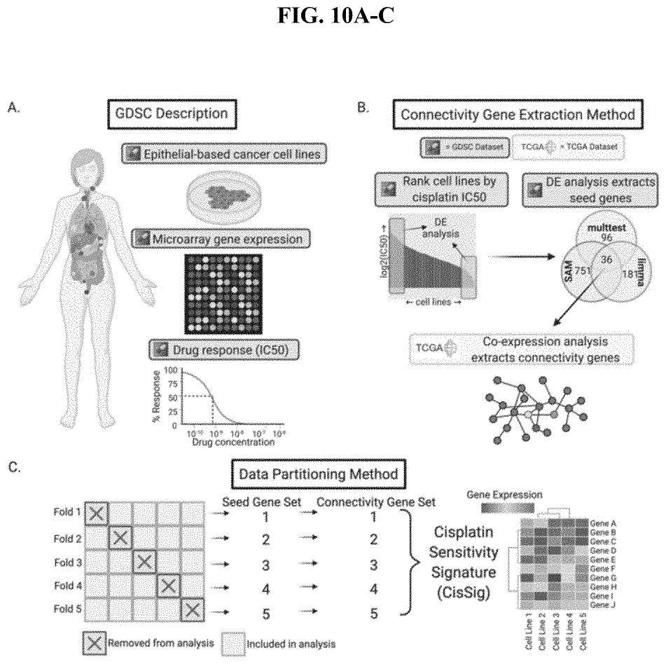

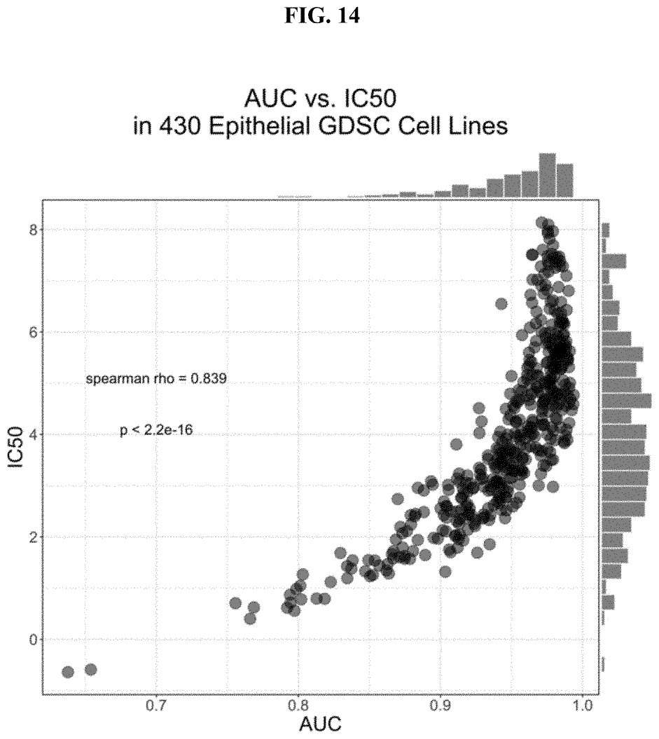

CisSig was derived using 429 epithelial-based cancer cell lines in the GDSC Database, each characterized for gene expression and drug response (see FIG. 10A). This database reports both IC50 and AUC as measures of drug response. A Spearman correlation between these two metrics demonstrated reasonable concordance (p=0.84, p<<0.001) in measuring cisplatin response for our cell lines of interest (FIG. 14). We therefore moved forward with IC50 as the metric of drug response, as it is a more commonly reported measure.

The GDSC epithelial cell lines were partitioned into five folds (each containing 343 or 344 cell lines) with a different 20% of the cell lines removed, illustrated in FIG. 10C. Each of these folds was analyzed with a pipeline of differential gene expression and co-expression analysis, visually depicted in FIG. 10B and discussed in detail below. This pipeline was performed across multiple partitions of the data in order to find genes that are consistent between folds, reducing the chance for outlier cell lines to influence the results.

Seed Genes are Extracted Using DE Analysis to Compare Cisplatin-Sensitive and -Resistant Cell Lines.

Cell lines with the highest and lowest 5% of IC50 values in each fold were removed in order to decrease the incidence of poorly modeled extreme drug responses from our analysis. Then, differential gene expression (DE) analysis using limma, 6 SAM, 7 and multtest 8 methods was performed between the top and bottom 20% of responders (i.e. cell lines with the highest and lowest 20% of IC50 values). For each fold, the genes over-expressed in a cisplatin-sensitive state by all three DE methods were termed the “seed genes,” resulting in 5 sets of seed genes, as depicted in FIG. 10C. Results of the DE analysis for each fold are summarized in Table 9.

TABLE 9
DE genes by fold. The SAM method consistently extracts more
genes than limma or multtest. The intersection, however, is much
smaller than either limma or multtest, showing significant
filtering during the intersection step.

| | | No. Up-regulated | No. Down-regulated |
|---|---|---|---|
| Fold | Method | Genes | Genes |
| 1 | SAM | 1979 | 1083 |
| | limma | 181 | 322 |
| | multtest | 219 | 150 |
| | intersection | 59 | 58 |
| 2 | SAM | 1397 | 853 |
| | limma | 159 | 302 |
| | multtest | 139 | 115 |
| | intersection | 32 | 41 |
| 3 | SAM | 2290 | 1143 |
| | limma | 176 | 355 |
| | multtest | 247 | 173 |
| | intersection | 58 | 73 |
| 4 | SAM | 1904 | 1069 |
| | limma | 188 | 263 |
| | multtest | 237 | 147 |
| | intersection | 61 | 42 |
| 5 | SAM | 566 | 636 |
| | limma | 156 | 221 |
| | multtest | 93 | 87 |
| | intersection | 34 | 28 |

Seed Genes are Used to Build Co-Expression Networks, which Inform the Final Signature.

A co-expression network was built for each set of seed genes, as described in Methods and visually represented in the bottom panel of FIG. 2B. These networks were built using The Cancer Genome Atlas (TCGA) RNA-Seq expression data from epithelial-based tumor samples, comparing the expression of each seed gene and all other genes in the dataset. Seed genes that were highly co-expressed with each other are extracted from each fold, termed “connectivity seeds.” For more details, see Methods. The final gene signature, CisSig, contains any gene found in at least 3 of the 5 sets of connectivity seeds, and the genes included in the signature are listed in Table 10.

TABLE 10
Genes included in CisSig. These genes all appear in at least 3 of the
5 sets of connectivity seeds.

| HGNC Gene | |
|---|---|
| Symbol | Gene Name |
| ADAT2 | Adenosine Deaminase tRNA Specific 2 |
| ATP1B3 | ATPase Na+/K+ transporting subunit beta 3 |
| CDIN1 | CDAN1 interacting nuclease 1 |
| C1QBP | Complement C1q binding protein |
| CDC7 | Cell division cycle 7 |
| CDCA7 | Cell division cycle associated 7 |
| FKBP14 | FKBP prolyl isomerase 14 |
| KRT5 | Keratin 5 |
| LRRC8C | Leucine rich repeat containing 8 VRAC subunit C |
| LY6K | Lymphocyte antigen 6 family member K |
| MMP10 | Matrix metallopeptidase 10 |
| NPM3 | Nucleophosmin 3 |
| PSAT1 | Phosphoserine aminotransferase 1 |
| RIOK1 | RIO kinase 1 |
| SLFN11 | Schlafen family member 11 |
| STOML2 | Stomatin like 2 |
| USP31 | Ubiquitin specific peptidase 31 |
| WDR3 | WD repeat domain 3 |
| ZNF750 | Zinc finger protein 750 |

sigQC is Used to Analyze the Quality of CisSig in a Clinical Dataset.

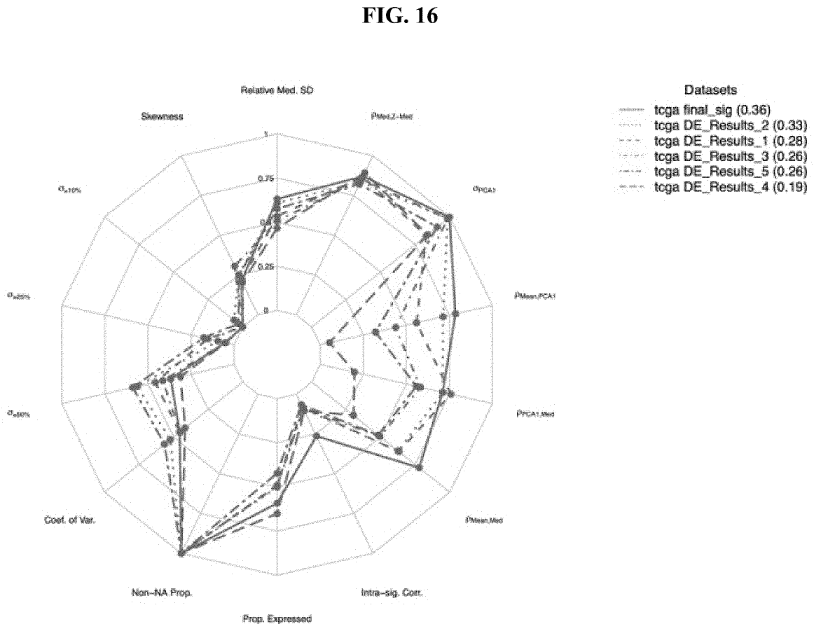

Using the ‘sigQC’ package in R, we analyzed a suite of quality control metrics to assess the robustness of CisSig in a clinical sample (TCGA) dataset. (9,10) The signature is compared to the 5 sets of seed genes originally extracted from GDSC, prior to being trimmed with co-expression analysis. These results are visualized in a radar plot in FIG. 16. CisSig demonstrates greater intra-signature correlation, increased correlation between mean and median, and decreased skewness within RNA-expression from TCGA samples of epithelial origin. Other metrics of interest include the coefficient of variance and the proportion (σ) of signature genes found in the top 10%, 25% or 50% of variable genes. These metrics can be used to assess the variability of signature genes within a dataset, where it is ideal to have signature genes that vary more than the background noise. Here, CisSig performs similarly to the unfiltered differential gene expression results. Finally, the these metrics are summarized into a score, also displayed in FIG. 16, where CisSig slightly outperformed all sets of seed genes.

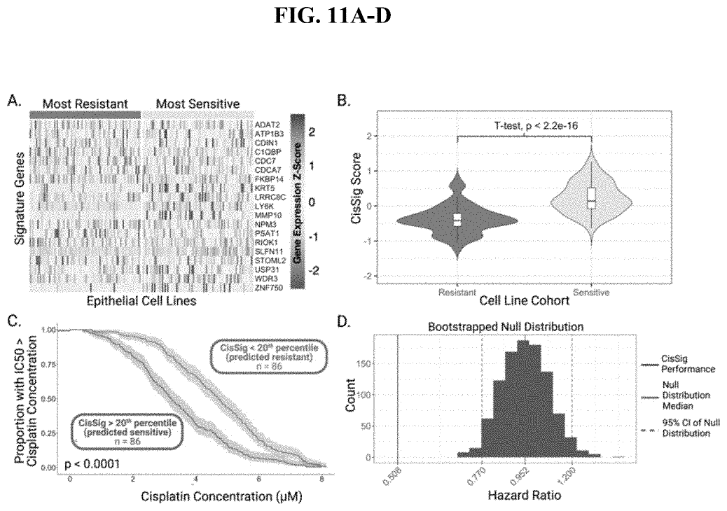

FIG. 11A demonstrates the expression of CisSig genes in cisplatin-sensitive and -resistant GDSC cell lines (top and bottom IC50) quintiles. From this, we see that signature expression tends to be higher (more red) in sensitive, rather than resistant, cell lines. Next, a “CisSig score,” the median normalized expression of the 19 CisSig genes, is calculated for the same sensitive and resistant cell lines. FIG. 11B shows that sensitive cell lines tend to have higher CisSig scores than resistant cell lines. This is expected, given that the seed genes were initially extracted as genes with increased expression in a cisplatin-sensitive state in the GDSC dataset.

FIG. 11C compares the distribution of IC50 between cohorts of GDSC cell lines in this top and bottom quintile of CisSig score. We are terming this plot a “Cell Line Persistence Curve”, which resembles a Kaplan-Meier survival curve, but uses IC50 in place of survival time for cell lines. Here, we assume that a cell line does not “survive” when the concentration of cisplatin is greater than it's IC50. For example, when cisplatin concentration reaches approximately 5 approximately 50% of the resistant cohort remains with a greater IC50, while only 20% of the sensitive cohort remains with a greater IC50. In other words, cell lines predicted to be resistant tend to have greater IC50 values and cell lines predicted to be sensitive tend to have lower IC50 values.

As demonstrated by Venet et al, many published gene signatures do not perform significantly better when predicting survival outcomes than random gene signatures of the same length 4. Given the large sample size of cell lines, simply testing for statistical significance may not be stringent enough. Therefore, we compared the performance of CisSig's Cell Line Persistence Curve (hazard ratio) to the performance of a null distribution. This null distribution was created using 1000 random gene signatures with the same length as CisSig, assessing the hazard ratio between each signature's Cell Line Persistence Curve. In FIG. 11D, we see that CisSig drastically outperforms the 95% confidence interval of this null distribution.

CisSig Outperforms the Null Distributions of Drug Response Prediction Models.

In order to further assess CisSig's predictive power within the GDSC dataset, a variety of prediction models were built using CisSig to predict IC50 of epithelial-based cell lines. Simple linear regression models used CisSig score to predict a cell line's IC50 as a continuous variable, while elastic net, L1-, and L2-penalized linear regression models used expression of all CisSig genes to predict a cell line's IC50 as a continuous variable. For these linear regression models, performance was compared using the Spearman correlation coefficient (ρ) between the predicted and actual IC50 value for the cell lines withheld from a given fold's training dataset. The best correlation coefficient between the five folds is chosen to represent each model, shown in Table 11.

TABLE 11
Model details and validation for the prediction of cisplatin response using CisSig in GDSC dataset.

| Input | Output | Method | Included Data | Metric | Value |
|---|---|---|---|---|---|
| Signature Score | IC50 (continuous) | Simple linear Regression | All | Corr. Coef. | 0.51 |
| Signature Score | IC50 (continuous) | Simple linear Regression | Quintiles | Corr. Coef. | 0.74 |
| All gene expression | IC50 (continuous) | Elastic Net Penalized Linear Regression | All | Corr. Coef. | 0.63 |
| All gene expression | IC50 (continuous) | Elastic Net Penalized Linear Regression | Quintiles | Corr. Coef. | 0.79 |
| All gene expression | IC50 (continuous) | L1-Penalized Linear Regression | All | Corr. Coef. | 0.63 |
| All gene expression | IC50 (continuous) | L1-Penalized Linear Regression | Quintiles | Corr. Coef. | 0.79 |
| All gene expression | IC50 (continuous) | L2-Penalized Linear Regression | All | Corr. Coef. | 0.63 |
| All gene expression | IC50 (continuous) | L2-Penatized Linear Regression | Quintiles | Corr. Coef. | 0.81 |
| All gene expression | Binary | Simple Logistic Regression | All | AUC | 0.79 |
| All gene expression | Binary | Simple Logistic Regression | Quintiles | AUC | 0.90 |
| All gene expression | Binary | Elastic Net Penalized Logistic Regression | All | AUC | 0.82 |
| All gene expression | Binary | Elastic Net Penalized Logistic Regression | Quintiles | AUC | 0.94 |
| All gene expression | Binary | L1-Penalized Logistic Regression | All | AUC | 0.82 |
| All gene expression | Binary | L1-Penalized Logistic Regression | Quintiles | AUC | 0.94 |
| All gene expression | Binary | L2-Penalized Logistic Regression | All | AUC | 0.81 |
| All gene expression | Binary | L2-Penalized Logistic Regression | Quintiles | AUC | 0.95 |
| All gene expression | Binary | Support Vector Machine (linear kernel) | All | AUC | 0.82 |
| All gene expression | Binary | Support Vector Machine (linear kernel) | Quintiles | AUC | 0.93 |
| All gene expression | Binary | Support Vector Machine (polynomial kernel) | All | AUC | 0.78 |
| All gene expression | Binary | Support Vector Machine (polynomial kernel) | Quintiles | AUC | 0.94 |
| All gene expression | Binary | Random Forest | All | AUC | 0.81 |
| All gene expression | Binary | Random Forest | Quintiles | AUC | 0.91 |

Simple logistic regression models used CisSig score to predict a cell line's IC50 as a binary outcome (above or below the median). Additionally, elastic net-, L1-, and L2-penalized logistic regression, support vector machine (with linear and polynomial kernels), and random forest models were built to use expression of each CisSig gene to predict IC50 as a binary outcome. We used area under the ROC curve (AUC) to represent each classification model's performance, again choosing the best of five folds to represent the model in Table 11.

In Table 11, we see that all models demonstrate improved performance when trained and tested on only cell lines with the highest and lowest signature scores (by quintile). Additionally, the penalized regression models outperform the simple regression models when comparing the same cell line data inputs.

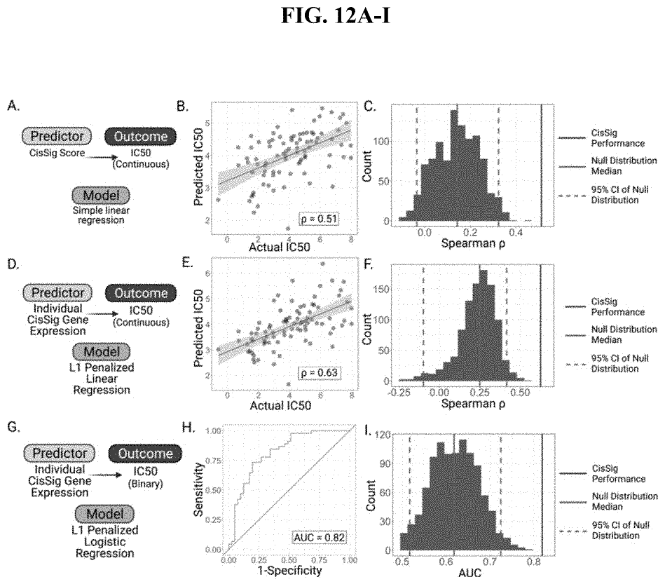

FIG. 12 shows three representative modeling methods, simple linear regression using CisSig score to predict continuous IC50 (FIGS. 12A-C), L1-penalized linear regression to predict continuous IC50 (FIGS. 12D-F), and L1-penalized logistic regression to predict binary IC50 (FIGS. 12G-I). These selected models include all epithelial-based cell lines in the GDSC dataset. For each method respectively, FIGS. 12B, 12E, and 12H demonstrate model performance in the validation cell lines from the best fold.

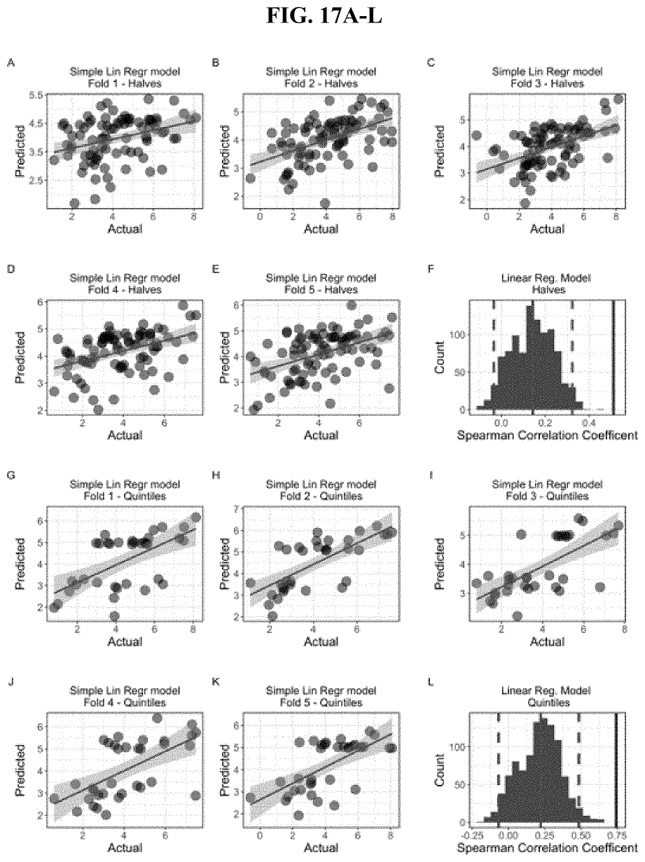

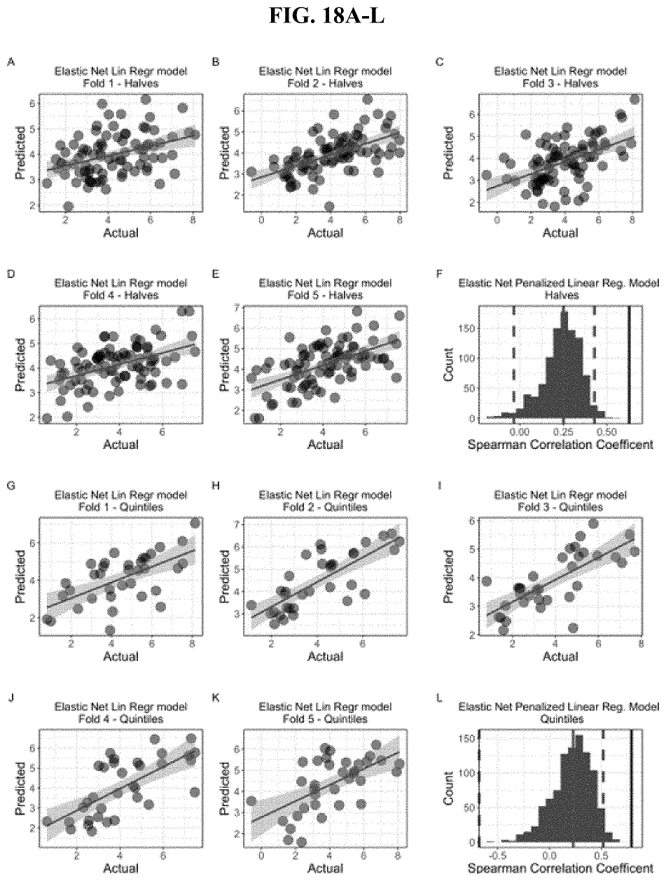

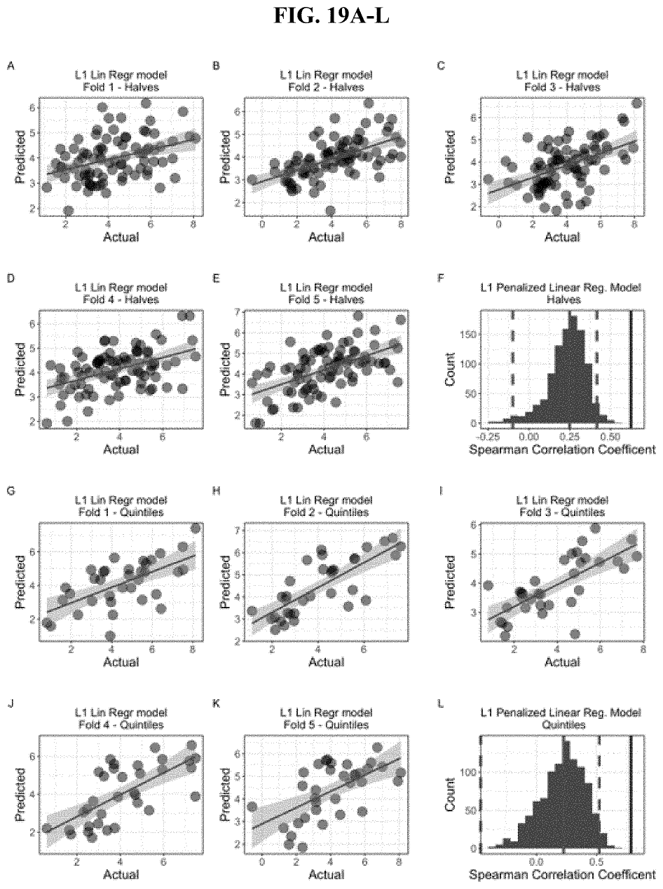

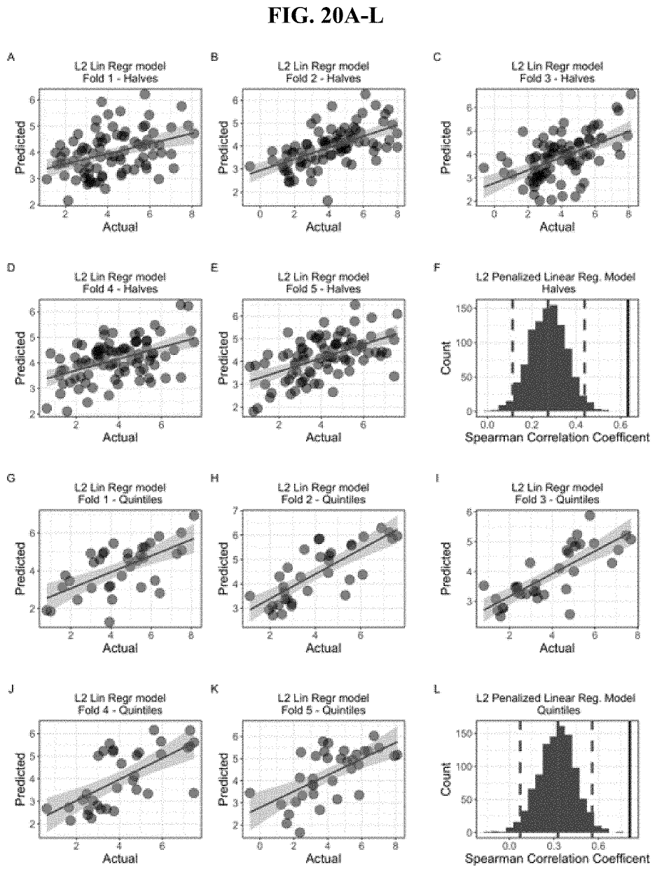

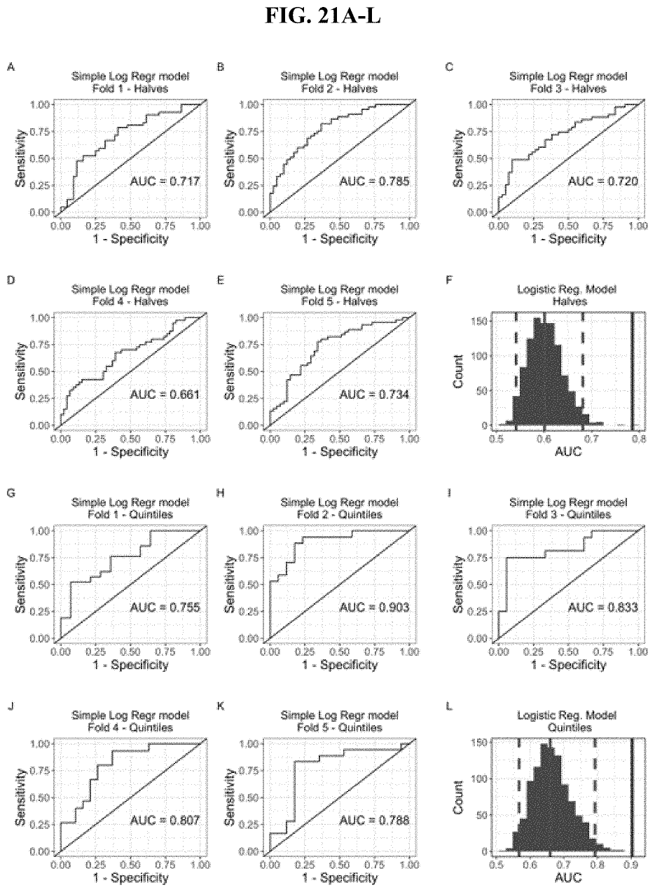

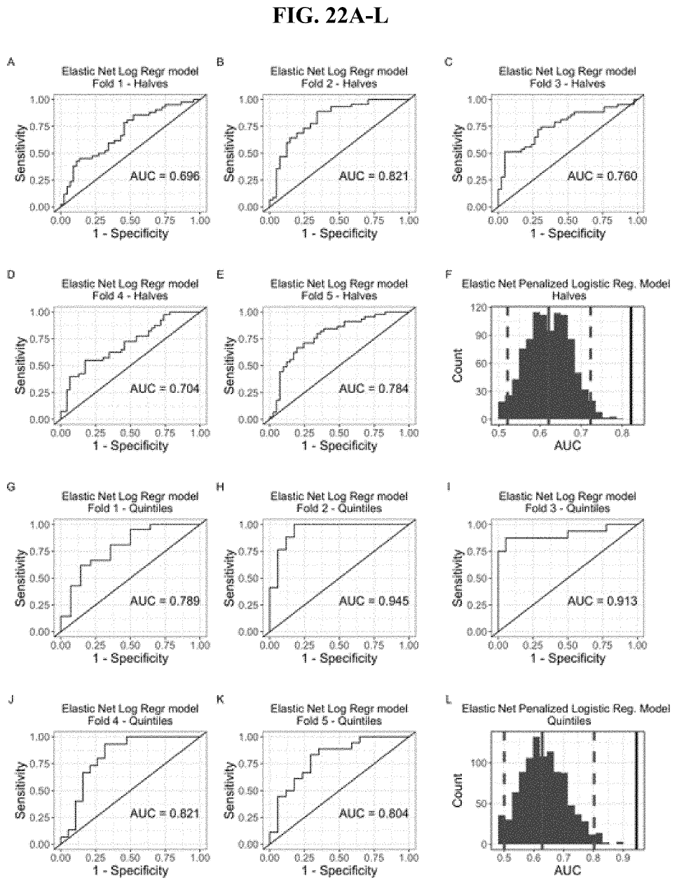

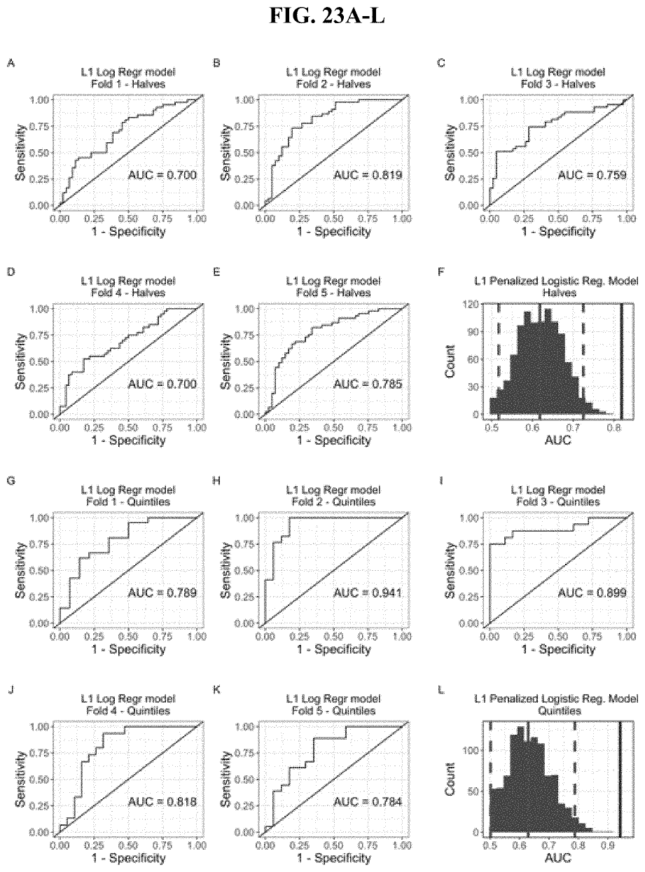

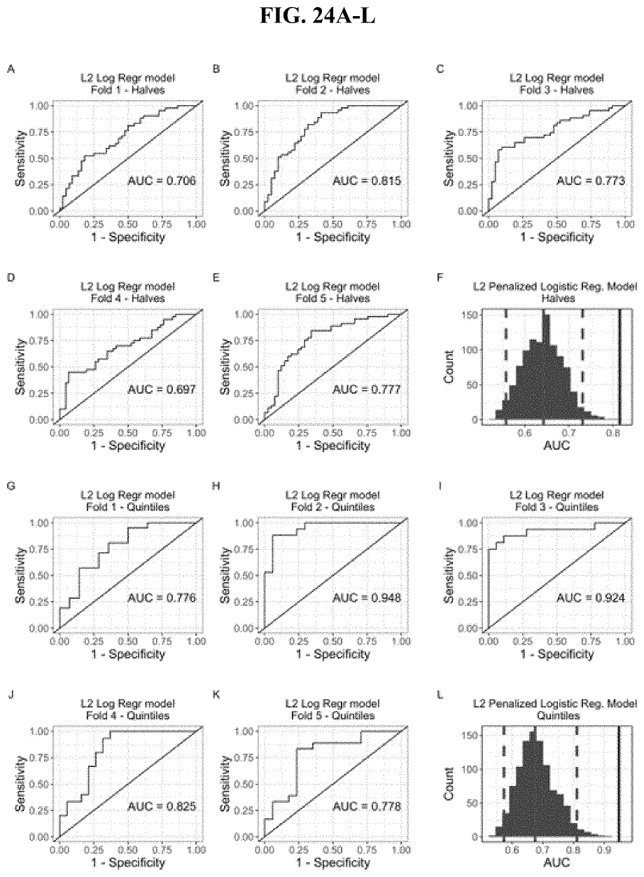

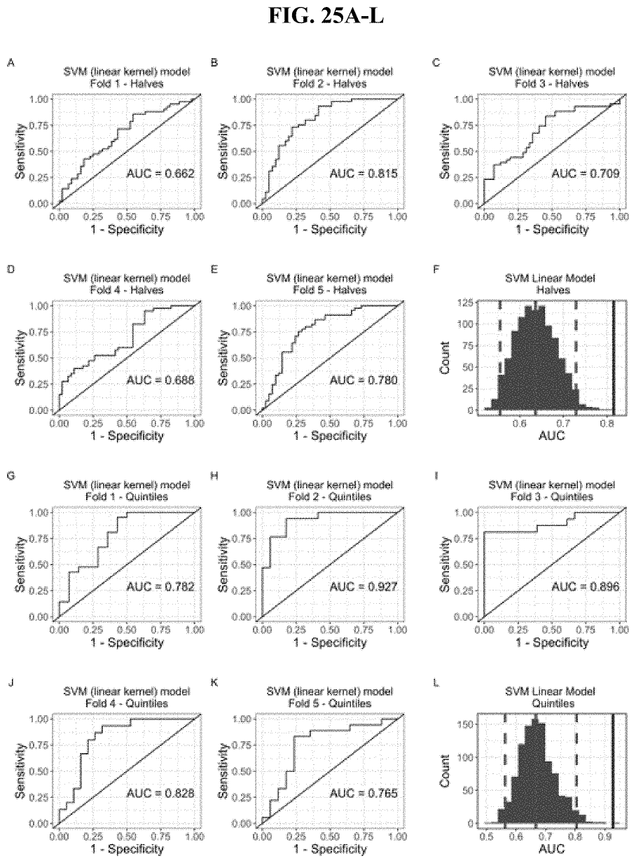

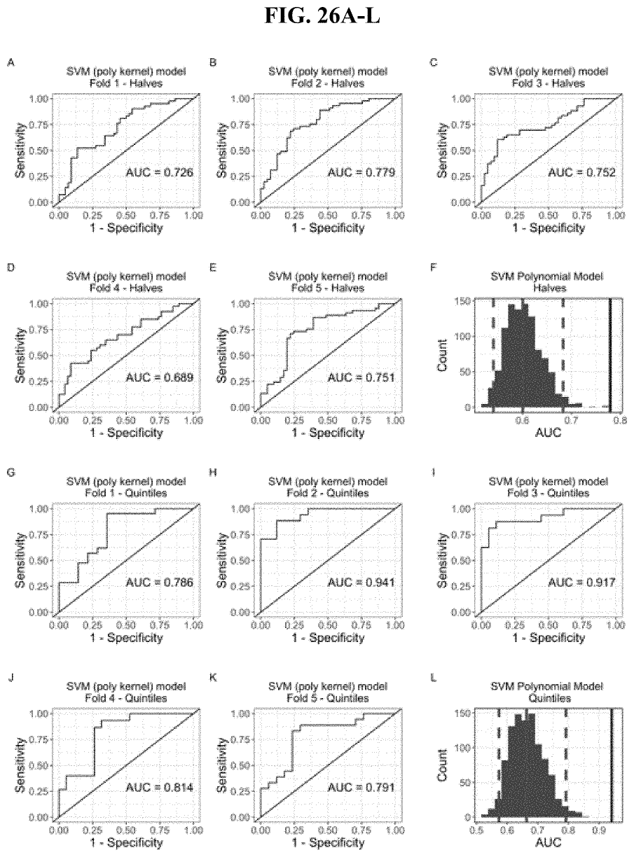

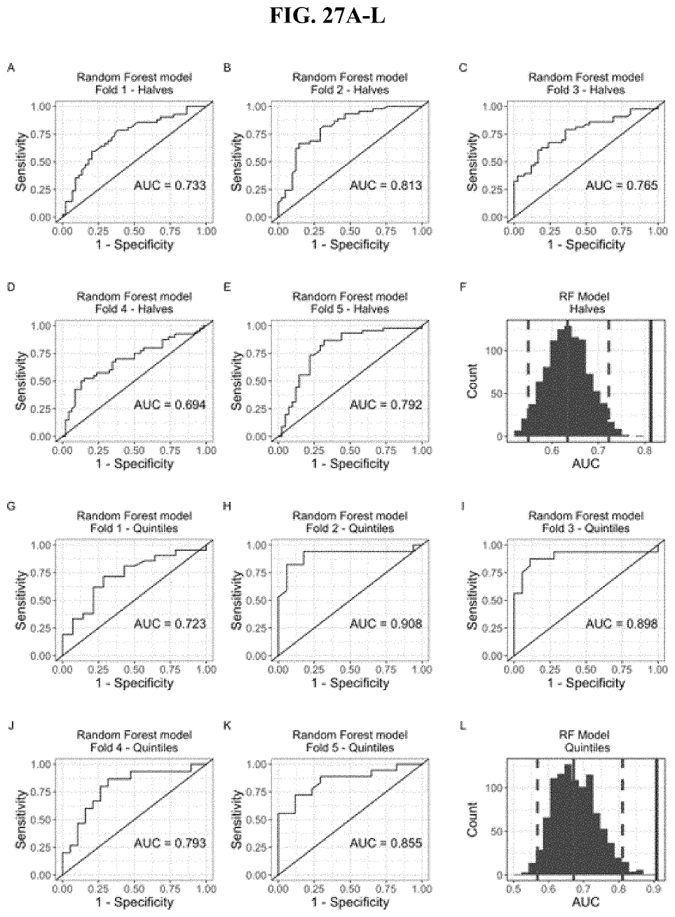

Similar to the null distribution for cell line persistence curves in FIGS. 12C-D, we also produced a bootstrapped null distribution for each model described in Table 11. Again, the null distribution consists of performance metrics from models built using 1000 random gene signatures each with 19 genes (the same length as CisSig) and five-fold cross validation. As with reporting model performance for CisSig, we chose the best metric of the 5 folds to represent the performance of each null signature. FIGS. 12C, 12F, and 12I visualize the null distributions for each method, where CisSig (blue line) outperforms the 95% confidence interval (dashed red lines) for each distribution. Finally, in FIGS. 17-27, we show that CisSig outperforms the 95% confidence interval of the null distributions for all models shown in Table 11.

Cancer Subtypes from Independent Clinical Data are Ranked by Signature Expression.

The consistently strong validation statistics displayed in FIGS. 11 and 12 demonstrate that this novel signature extraction methodology is capable of selecting genes with strong predictive power within the dataset of extraction. In other words, it is a powerful tool for feature selection. In order to assess translation into novel datasets; however, predictive power should generally be demonstrated in datasets that were not used to select genes of interest.

Using three large datasets, we assessed how expression of CisSig relates to cisplatin use across epithelial-based cancer disease sites. CisSig score was calculated for all samples (cell lines or clinical tumor samples) in GDSC, TCGA, and TCC databases. In order to visualize these scores on a log-transformed axis, signature score was linearly scaled, making the lowest score exactly 1.

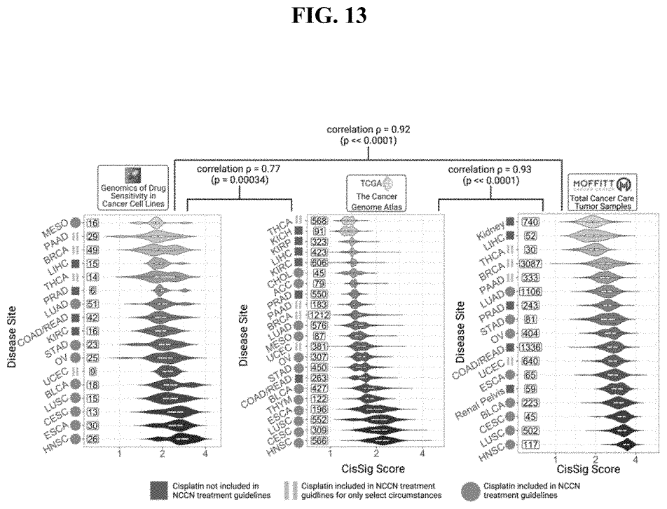

In FIG. 13, disease sites were ranked by the median signature score for the cohort in GDSC (left), TCGA (middle), and TCC (right) datasets. Furthermore, each disease site is labeled as utilizing cisplatin in NCCN treatment guidelines, using cisplatin in very select circumstances, or not having cisplatin included in NCCN treatment guidelines. In all datasets, we see that disease sites with higher CisSig scores tend to have cisplatin included in treatment guidelines, while those with lower scores tend to not have cisplatin included in treatment guidelines. Consistent between all three datasets, CisSig scores were highest for head-neck squamous cell carcinoma and cervical cancer.

Finally, disease site rank was compared between datasets using Spearman correlation. In FIG. 13, we see that disease site ranking in GDSC (cell line database) was moderately correlated to TCGA and TCC (both clinical tumor sample databases). Meanwhile, there was a strong correlation between the two clinical sample databases, TCGA and TCC.

Methods

Data Collection and Pre-Processing

All data cleaning, analysis, and plotting was performed using R with RStudio.

GDSC Gene Expression Data

Microarray mRNA expression, drug response, and meta-data for 983 cell lines and 251 drugs was downloaded from the Genomics in Drug Sensitivity Database (GDSC)(13). The expression and meta-data were last updated 4 Jul. 2016. The GDSC database can be accessed at https://www.followed by cancerrxgene.org/. Documentation for the GDSC database states that the RMA normalized14 expression data for all cell lines were collected via Affymetrix Human Genome U219 Array. The raw data and probe ID mappings were deposited in ArrayExpress (accession number: E-MTAB-3610). The RMA processed dataset is available at http://www.followed by cancerrxgene.org/gdsc1000/.

Epithelial-based cell lines are extracted based on the following GDSC tissue descriptors: “head and neck”, “oesoph-agus”, “breast”, “biliary_tract”, “digestive_system_other”, “large_intestine”, “stomach”, “lung_NSCLC_adenocarcinoma”, “lung_NSCLC_carcinoid”, “lung_NSCLC_large cell”, “lung_NSCLC_not specified”, “lung_NSCLC_squamous_cell_carcinoma”, “Lung_other, pancreas”, “skin_other”, “thyroid”, “Bladder”, “cervix”, “urogenical_system_other”, and “uterus”.

GDSC Drug Response Data

The drug response data in the GDSC database was last updated 27 Mar. 2018; this version is referred to as “GDSC2.” Cisplatin drug concentration is reported in μM. Raw viability data were processed using the R package, gdscIC50, where they were normalized with negative controls (media alone) and positive controls (media only wells with no cells). Dose-response curves were fit using a multi-level fixed effect model with a classic sigmoidal curve shape assumed. This model was fitted using all cell line/drug combinations that were screened instead of fitting separate models to individual drug-response series. In this approach, the shape parameter only changes between cell lines, but the position parameter is adjusted between cell lines and compounds. Additional information regarding dose-response curve fitting may be found at Vis et al. 15. Fitting models to all dose-response series leads to improved robustness for more accurate IC50 and AUC estimates.

TCGA Gene Expression Data

RSEM normalized gene expression for epithelial-based cancers was downloaded from The Cancer Genome Atlas (TCGA) database, which was accessed through the Firebrowse database at http://www.firebrowse.org. The following TCGA Study Abbreviations were downloaded: “BLCA”, “BRCA”, “CESC”, “COAD”, “HNSC”, “KIRP”, “LGG”, “KIRC”, “LIHC”, “LUAD”, “LUSC”, “OV”, “PAAD”, “PRAD”, “STAD”, “THCA”, and “UCEC.” These values were taken through the Illumina HiSeq RNAseq V2 platform and were log 2 transformed.

Total Cancer Care (TCC) Gene Expression Data

The Total Cancer Care Dataset is collected by the H. Lee Moffitt Cancer Center and Research Institute using protocols described in Fenstermacher et al (16).

Drug Response Quality Control

IC50 is an imperfect measure of drug response, yet it is widely used throughout the literature. IC50 and AUC values for all epithelial cell lines are compared using a Spearman correlation test (see FIG. 14) in order to assess concordance between the two metrics.

Differential Gene Expression Analysis

As seen in FIG. 10C, the GDSC dataset is split into 5-folds, where 20% of the cell lines are removed from further analysis for each of the 5 runs. After data partitioning, the top 20% and bottom 20% are extracted for comparison using differential expression analysis, FIG. 10C. Differential expression analysis is performed using three algorithms, significance analysis of microarrays (SAM), resampling-based multiple hypothesis testing, and linear models for microarrays (limma), which are implemented using R packages samr7, multtest8, and limma6, respectively. This analysis has 62 samples per group, which is appropriate given the demonstration by Baccarella et al. showing that differential expression results begin to vary problematically beginning when there are as few as 8 samples per group 17. A false discovery rate of 0.20 was chosen for each method. The intersection of the genes found to have significantly increased expression in sensitive cell lines by the three algorithms is termed “seed genes” for use in future co-expression analysis.

Co-Expression Network Analysis and Final Signature Derivation

The co-expression network, represented in the pipeline of FIG. 10B, is made by performing a pairwise Spearman correlation between the expression of each seed gene and every other gene (including other seed genes) except itself. The correlation coefficient for each pairwise comparison is termed the “affinity score.” Next, the network is transformed so that the largest 5% of affinity scores are transformed to 1 and all other scores become 0. This is done without squaring the scores in order to extract only positive correlations. The average affinity score for each gene compared to each seed gene is then derived; this value becomes known as a gene's “connectivity score.” The intersection between the differentially expressed seed genes and genes with the top 20% of the highest connectivity scores become known as the “connectivity genes.” Five sets of connectivity genes are compiled, one for each data partition. The final signature (CisSig) is produced by extracting any gene that is found in at least three of the five connectivity gene sets.

Signature Quality Control in TCGA

In order to examine how CisSig compares to the original differential gene expression results, we perform a quality control analysis within the TCGA dataset using the sigQC R package. (9,10). Here, various metrics are calculated using the expression of the genes found in the gene expression signature and the 5 sets of differential expression analysis results. These metrics include intra-signature correlation, correlation between the mean expression and first principal component, and skewness of the signature expression. The final results of all the metrics calculated for each signature are displayed in a radar plot, with a summary score of each set of genes (signature) tested. This summary score is the ratio of the area within the radar plot and the full polygon if each metric was the highest value possible. For more details on sigQC, please see Dhawan et al, 2019. (10).

Modeling Cell Line IC50 in GDSC

A cell line or sample's median normalized expression value of the CisSig genes is termed the CisSig score. Cell lines were again organized into five folds (independent of the data partitioning used in the signature extraction, described in FIG. 10C). Predictive models were built using 80% of the cell lines (training cell lines) and tested on the 20% of the cell lines withheld from the model (validation cell lines). All models were built with two versions of input—one using all of the epithelial-based cell lines in the GDSC database and the other using only the cell lines in the top and bottom quintiles of CisSig score. When using all the epithelial-based cell lines, training sets consist of 344-345 cell lines, while testing sets consist of 86 cell lines. When using only the cell lines in the top and bottom quintiles for signature expression, training sets consist of 137 or 138 cell lines and testing sets consist of 34 or 35 cell lines.

Linear and logistic regression was used to predict IC50 given a cell line's CisSig score. Elastic net, L1-, and L2-penalized linear and logistic regression, support vector machine (SVM), and random forest methods utilized the expression of each of the 19 CisSig genes to predict IC50. Linear regression models used IC50 as a continuous outcome variable, and were evaluated using the Spearman correlation coefficient between true and predicted IC50 values from the validation set. Classification models (logistic regression, SVM, and random forest) used IC50 as a binary outcome variable (above or below median IC50 of the group), and were evaluated using area under the receiver operating characteristic (ROC) curve (AUC).

Elastic net, L1-, and L2-penalized linear and logistic regression models were built using the ‘glmnet’ package in R. The alpha parameter was set to 0.5, 1, and 0 for elastic net, L1-, and L2-penalized regression, respectively. Models were tuned with 10-fold cross validation to choose a value for lambda with the best predictive capabilities based on mean square error for linear models and misclassification error for logistic models.

SVM models were built with the ‘e1071’ package in R, using both a linear and polynomial kernel. Models were tuned with 10-fold cross validation to choose the best value for degree (from 3, 4, 5), gamma (from 10-3, 10-2, 10-1, 1, 101, 102, 103), and cost (from 10-3, 10-2, 10-1, 1, 101, 102, 103).

The random forest model grew 500 trees. All other parameters in training the prediction models were default. The code for building all of these models can be viewed in the GitHub repository listed in Code and Data Availability. Each of these classification algorithms was evaluated using the area under the curve (AUC) value for the receiver operating characteristics (ROC) curve for each model.

Null Distributions of Cell Line IC50 Models

For each model, 1000 random gene signatures of the same length are tested to produce a null distribution of these summary statistics. The null models are built using random gene signatures of the same length as CisSig (19 genes). Just as seen when modeling drug response with CisSig, each null gene signature is tested using five-fold cross validation for each modeling method and the best summary statistic of the five-folds is chosen to represent the signature's performance with a given model. Again, all code for building the testing and null models may be found in the GitHub repository listed in Code and Data Availability.

Pseudo-Kaplan-Meier Statistics

Cell lines with high CisSig scores (predicting the more sensitive cell lines) and low signatures scores (predicting the more resistant cell lines) are separated by quintile. A Kaplan-Meier survival model is built for the two cohorts using IC50 in lieu of survival time. A log-rank test compares the two survival curves to analyze if the two cohorts of signature expression are related to different “survival” of higher IC50s in each group. Again, a null distribution was built using 1000 random gene signatures of the same length as CisSig.

Ranking Disease Sites in GDSC, TCGA, and TCC by CisSig Score

All epithelial-origin cell lines or tumor samples in the GDSC, TCGA, and TCC datasets had CisSig Score calculated as previously described. For the purposes of plotting on a log-scale, the scores were linearly adjusted so the lowest score became Disease sites within each dataset were ranked by median CisSig score. For disease sites shared between datasets, a Spearman correlation was performed to assess how the disease sites rank.

NCCN Treatment Guidelines for each disease site were manually searched, versions listed in Table 12. Disease sites were classified as including cisplatin in treatment guidelines, only including cisplatin in very select circumstances, or not including cisplatin in treatment guidelines. For those classified as only using cisplatin in select circumstances, details are noted in Table 12.

TABLE 12
NCCN Guideline versions used for assessing disease-site specific treatment guidelines.

| Disease | | | |
|---|---|---|---|
| Site | NCCN Guideline Version | Cisplatin Use | Notes for select circumstances |
| ACC | Neuroendocrine and Adrenal Tumors Version | Yes | |
| | 3.2021 | | |
| BLCA | Bladder Cancer Version 3.2021. | Yes | |
| BRCA | Breast Cancer Version 5.2021 | Select circumstances | Only for recurrent unresectable |
| | | | triple negative BRCA with |
| | | | germline BRCA1/2 mutation |
| CESC | Cervical Cancer Version 1.2021 | Yes | |
| CHOL | Hepatobiliary Cancers Version 5.2021 | Yes | |
| COAD | Colon Cancer Version 2.2021 | No | |
| ESCA | Esophageal and Esophagogastric Junction Can- | Yes | |
| | cers Version 3.2021 | | |
| HNSC | Head and Neck Cancers Version 3.2021 | Yes | |
| KICH | Kidney Cancer Version 2.2022 | No | |
| KIRP | Kidney Cancer Version 2.2022 | No | |
| KIRC | Kidney Cancer Version 2.2022 | No | |
| Kidney | Kidney Cancer Version 2.2022 | No | |
| Renal | Kidney Cancer Version 2.2022 | No | |
| Pelvis | | | |
| LIHC | Hepatobiliary Cancers Version 3.2021 | No | |
| LUAD | Non-Small Cell Lung Cancer Version 5.2021 | Yes | |
| LUSC | Non-Small Cell Lung Cancer Version 5.2021 | Yes | |
| MESO | Malignant Pleural Mesothelioma Version 2.2021 | Yes | |
| OV | Ovarian Cancer/Fallopian Tube Cancer/ Primary | Yes | |
| | Peritoneal Cancer Version 1.2021 | | |
| PAAD | Pancreatic Adenocarcinoma Version 2.2021 | Select circumstances | Only for BRCA1/2 or PALB2 |
| | | | mutations |
| PRAD | Prostate Cancer Version 2.2021 | No | |
| READ | Rectal Cancer Version 1.2021 | No | |
| STAD | Gastric cancer Version 3.2021 | Yes | |
| THCA | Thyroid Carcinoma Version 1.2021 | Select circumstances | Only as adjuvant/radiosensitizer |
| | | | for anaplastic carcinoma |
| THYM | Thymomas and Thymic Carcinomas Version | Yes | |
| | 1.1021 | | |
| UCEC | Uterine Neoplasms Version 3.2021 | Yes | |

### Example 3

### CisSig is Predictive of Survival in Muscle-Invasive Bladder Cancer Patients Who Received Cisplatin Containing Chemotherapy

We trained and tested a Cox proportional hazards (PH) survival model using CisSig genes (Table 10 above) in two publicly available datasets described in in Table 13. Within Dataset A, we performed univariate survival analysis with each of the CisSig genes using only samples that received cisplatin-containing neo-adjuvant chemotherapy. Genes with a strong relationship between increased expression and improved survival were selected to be included in multivariate analysis, described in detail in Methods.

TABLE 13
Description of clinical datasets used for training and testing of
CisSig-informed survival model. Treatment refers to neoadjuvant
MVAC chemotherapy, which is a regiment that includes
methotrexate, vinblastine, doxorubicin, and cisplatin.

| | GSE Accession | Disease | n with | n without |
|---|---|---|---|---|
| Name | No. | Site | treatment | treatment |
| Dataset A | G5E45276 | Bladder | 16 | 37 |
| Dataset B | GSE70691 | Bladder | 22 | 0 |

Methods

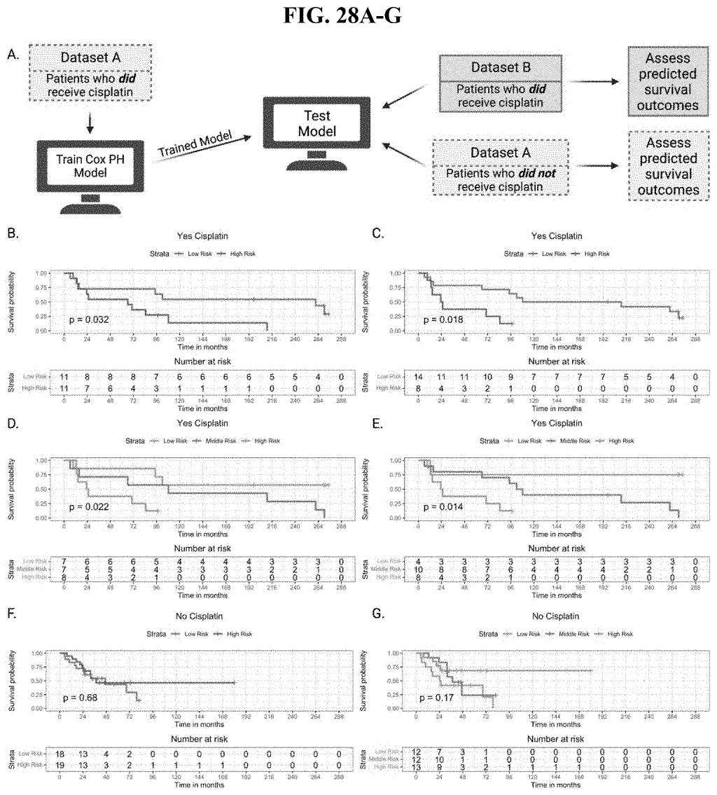

Survival Analysis in External MIBC Cohorts Two separate models were trained, using a similar method displayed in FIG. 28A and

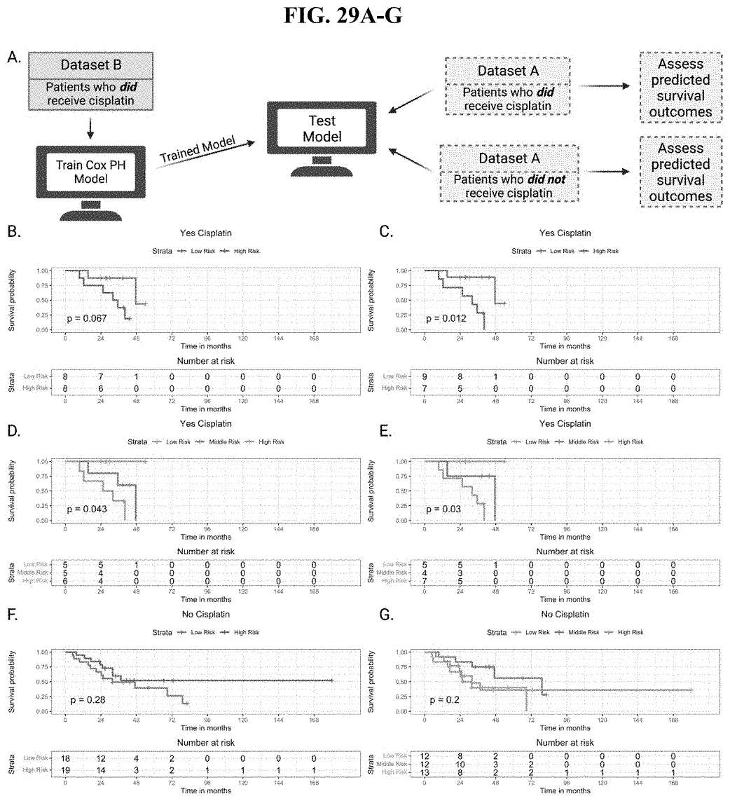

FIG. 29A, respectively. For the model trained in FIG. 29A, we performed univariate analysis for each CisSig gene to predict overall survival of samples that received cisplatin-containing chemotherapy in Dataset A. A multivariate model was trained using genes from the univariate analysis that demonstrated a coefficient of −0.5 or lower; this became the trained model. Both univariate and multivariate models were build using the ‘Sure’ and ‘coxph’ function from the ‘survival’ package in R. The trained model was tested using the ‘predict’ function in R, extracting the linear predictor for samples from Dataset B who received cisplatin-containing neoadjuvant chemotherapy and samples from Dataset A who did not receive any cisplatin-containing treatment. Samples were separated by median, optimal single cutpoint, tertiles, and optimal double cutpoints. Cohorts separated by each cutpoint were compared using Kaplan-Meier analysis, using the ‘ggsurvplot’ function in the ‘survminer’ package in R. The same analysis was performed for FIG. 29, except the training dataset was Dataset B (patients who received cisplatin-containing neoadjuvant chemotherapy), while the testing datasets were patients from Dataset A who did and did not receive cisplatin-containing neoadjuvant chemotherapy.

As shown in FIG. 28A, this multivariate analysis used Dataset A samples that received cisplatin-containing treatment, produced a trained Cox PH model. We tested this model using samples from Dataset B, all of which received cisplatin-containing chemotherapy, and samples from Dataset A, all of which did not receive cisplatin-containing chemotherapy. FIGS. 28B-C show that samples predicted to be “high risk” have significantly worse survival than patients predicted to be “low risk.” FIG. 28B uses an arbitrary cutoff (median) to separate the cohorts, while FIG. 28C uses the optimal cutoff to separate the groups.

Similarly, FIGS. 28D-E show significant separate between “high”, “medium”, and “low risk” cohorts with worst to best survival outcomes, respectively. Again, FIG. 28D uses an arbitrary cutoff (tertiles) to separate the cohorts, while FIG. 28E uses the optimal two cutpoints for each cohort. Finally, FIGS. 6F-G show that the signal is lost when testing our model with either binary or tertile cohorts in patients from Dataset A who did not receive cisplatin-containing chemotherapy. The reverse of these analyses, where the model is trained with Dataset B's patients who did receive cisplatin-containing chemotherapy, then is tested using Dataset A's patients who both did and did not receive cisplatin-containing chemotherapy shows the same results, shown in FIG. 29. The coefficients and their standard errors can be found in Tables 14 and 15.

TABLE 14
Resulting coefficients for multivariate model trained in FIG. 28.

| | Coefficient | Exponentiated Coefficient | Coefficient Standard |
|---|---|---|---|
| Gene | Estimate | Estimate | Error |
| C15orf41 | −2.2185 | 0.1088 | 1.3481 |
| FKBP14 | −1.5948 | 0.2029 | 1.0083 |
| PSAT1 | −1.8362 | 0.1594 | 1.5889 |
| C1QBP | 0.2797 | 1.3227 | 1.0234 |

TABLE 15
Resulting coefficients for multivariate model trained in FIG. 29.

| | Coefficient | Exponentiated Coefficient | Coefficient Standard |
|---|---|---|---|
| Gene | Estimate | Estimate | Error |
| C15orf41 | −0.6801 | 0.5066 | 0.3784 |
| FKBP14 | −0.3735 | 0.6883 | 0.5040 |
| PSAT1 | −1.2059 | 0.8139 | 0.5684 |

### REFERENCES

1. Marquart, J., Chen, E. Y. & Prasad, V. Estimation of the percentage of us patients with cancer who benefit from genome-driven oncology. JAMA oncology 4, 1093-1098 (2018).
2. Sparano, J. A. et al. Adjuvant chemotherapy guided by a 21-gene expression assay in breast cancer. New Engl. J. Medicine 379, 111-121 (2018).
3. Soliman, H. et al. Mammaprint guides treatment decisions in breast cancer: results of the impact trial. BMC cancer 20, 81 (2020).
4. Venet, D., Dumont, J. E. & Detours, V. Most random gene expression signatures are significantly associated with breast cancer outcome. PLoS computational biology 7, e1002240 (2011).
5. Buffa, F., Harris, A., West, C. & Miller, C. Large meta-analysis of multiple cancers reveals a common, compact and highly prognostic hypoxia metagene. Br. journal cancer 102, 428 (2010).
6. Yang, W. et al. Genomics of drug sensitivity in cancer (gdsc): a resource for therapeutic biomarker discovery in cancer cells. Nucleic Acids Res. 41, D955-D961.
7. Ritchie, M. E. et al. limma powers differential expression analyses for ma-sequencing and microarray studies. Nucleic Acids Research 43, e47-e47 (2015).
8. Tusher, V., Tibshirani, R. & Chu, C. Significance analysis of microarrays applied to ionizing radiation response. Proc. Natl. Acad. Sci. 98, 5116-5121 (2001).
9. Pollard, K. S., Dudoit, S. & van der Laan, M. J. Multiple testing procedures: the multtest package and applications to genomics. In Bioinformatics and computational biology solutions using R and bioconductor, 249-271 (Springer, 2005).
10. Dhawan, A., Barberis, A., Cheng, W.-C. & Buffa, F. sigQC: Quality Control Metrics for Gene Signatures (2018). R package version 0.1.21.
11. Dhawan, A. et al. Guidelines for using sigqc for systematic evaluation of gene signatures. Nat. Protoc. 14, 1377 (2019).
12. Azuaje, F. Computational models for predicting drug responses in cancer research. Briefings bioinformatics 18, 820-829 (2017).
13. Goodspeed, A., Heiser, L. M., Gray, J. W. & Costello, J. C. Tumor-derived cell lines as molecular models of cancer pharmacogenomics. Mol. Cancer Res. 14, 3-13 (2016).
14. Irizarry, R. A. et al. Exploration, normalization, and summaries of high density oligonucleotide array probe level data. Biostatistics 4, 249-264 (2003).
15. Vis, D. J. et al. Multilevel models improve precision and speed of ic50 estimates. Pharmacogenomics 17, 691-700 (2016).
16. Durinck, S., Spellman, P. T., Birney, E. & Huber, W. Mapping identifiers for the integration of genomic datasets with the r/bioconductor package biomart. Nat. protocols 4, 1184 (2009).
17. Durinck, S. et al. Biomart and bioconductor: a powerful link between biological databases and microarray data analysis. Bioinformatics 21, 3439-3440 (2005).
18. Fenstermacher, D. A., Wenham, R. M., Rollison, D. E. & Dalton, W. S. Implementing personalized medicine in a cancer center. Cancer journal (Sudbury, Mass.) 17, 528 (2011).
19. Baccarella, A., Williams, C. R., Parrish, J. Z. & Kim, C. C. Empirical assessment of the impact of sample number and read depth on rna-seq analysis workflow performance. BMC bioinformatics 19, 423 (2018).

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method comprising:
a) receiving results of, or conducting, an mRNA or protein expression level analysis of at least one gene from epithelial tumor cells from a subject, wherein said at least one gene mRNA and/or protein is expressed at higher levels compared to said at least one gene mRNA and/or protein expression from corresponding non-tumor epithelial cells,
wherein said at least one gene is selected from the group consisting of: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750; and
b) performing at least one of the following:
i) treating said subject with Cisplatin or other platinum based cancer drug, and/or
ii) providing a report to said patient or medical personnel treating said patient, indicating said subject is suitable for, or should be, treated with Cisplatin or other platinum based cancer drug.

2. The method of claim 1, further comprising: receiving results of, or conducting, an mRNA or protein expression level analysis of at least two genes from epithelial tumor cells from a subject, wherein said at least two genes are expressed at higher levels compared said at least two genes from corresponding non-tumor epithelial cells, wherein said at least two genes are selected from the group consisting of: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750.

3. The method of claim 2, wherein said at least two genes is at least three to at least thirteen genes.

4. The method of claim 2, wherein said at least two genes is at least thirteen genes that includes all of the following: ADA, NPM3, CSTA, KRT5, KRT14, ATP1B3, USP31, MAP7D3, LRRC8C, C15orf41, LY6K, BNC1, and SLFN11.

5. The method of claim 2, wherein said at least two genes are selected from the group consisting of: NPM3, KRT5, ATP1B3, USP31, LRRC8C, LY6K, and SLFN11.

6. The method of claim 1, wherein said at least two genes includes the following 7 genes: NPM3, KRT5, ATP1B3, USP31, LRRC8C, LY6K, and SLFN11.

7. The method of claim 2, wherein said at least two genes is at least three to at least nineteen genes.

8. The method of claim 2, wherein said at least two genes is at least nineteen genes that includes all of the following: NPM3, KRT5, ATP1B3, USP31, LRRC8C, LY6K, SLFN11, ADAT2, CDIN1, C1QBP, CDC7, CDCA7, FKBP14, MMP10, PSAT1, RIOK1, STOML2, WDR3, and ZNF750.

9. The method of claim 1, wherein at least one gene comprises: C15orf41, FKBP14, and PSAT1.

10. The method of claim 1, wherein said at least said at least one gene comprises: C15orf41, FKBP14, PSAT1, and C1QBP.

11. The method of claim 1, wherein said subject is a human with cancer.

12. The method of claim 11, wherein said cancer comprises muscle-invasive bladder cancer.

13. The method of claim 1, wherein said method comprises receiving results of conducting an mRNA expression level analysis.

14. The method of claim 1, wherein said method comprises conducting an mRNA expression level analysis.

15. The method of claim 14, wherein said detecting comprises the use of one or more nucleic acid reagents selected from the group consisting of a nucleic acid primers and nucleic acid probes.

16. The method of claim 1, wherein said method comprises conducting protein expression level analysis.

17. The method of claim 16, wherein said detecting comprises the use of one or more antibodies or antigen binding fragments thereof.

18. A kit for detecting altered levels of gene mRNA and/or protein expression in a sample from a subject, comprising:
reagents that specifically detect mRNA and/or protein expression from two or more genes selected from the group consisting of: ADA, NPM3, CSTA, KRT5, KRT14, ATP1B3, USP31, MAP7D3, LRRC8C, C15orf41, LY6K, BNC1, SLFN11, ADAT2, CDIN1, C1QBP, CDC7, CDCA7, FKBP14, MMP10, PSAT1, RIOK1, STOML2, WDR3, and ZNF750.

19. The kit of claim 18, wherein said reagents are selected from the group consisting of nucleic acid primers, nucleic acid probes, and antibodies or antigen binding fragments thereof.

20. The kit of claim 18, wherein said two or more genes comprises: C15orf41, FKBP14, and PSAT1.
